(19) United States
(12) Patent Application Publication
(10) Pub. No.: US 2019/0269797 A1
(43) Pub. Date: Sep. 5, 2019

(54) GENE TRANSFER COMPOSITIONS, METHODS AND USES FOR TREATING NEURODEGENERATIVE DISEASES

(71) Applicant: The Children's Hospital of Philadelphia, Philadelphia, PA (US)

(72) Inventors: Beverly L. DAVIDSON, Philadelphia, PA (US); Yong Hong CHEN, Philadelphia, PA (US); Luis TECEDOR, Philadelphia, PA (US)

(73) Assignee: The Children's Hospital of Philadelphia, Philadelphia, PA (US)

(21) Appl. No.: 16/344,298

(22) PCT Filed: Nov. 3, 2017
(86) PCT No.: PCT/US2017/059986

§ 371 Date: Apr. 23, 2019

Related U.S. Application Data
(60) Provisional application No. 62/418,033, filed on Nov. 4, 2016.

(51) Int. Cl. A61K 48/00; C12N 15/86; A61K 38/48; A61K 38/13; A61K 31/365

(52) U.S. Cl. CPC A61K 48/0066; C12N 15/86; A61K 38/4813; C12Y 304/14009; C12N 2750/14122; A61K 38/13; A61K 31/365; C12N 2750/14143; A61K 48/0058

(57) ABSTRACT

Provided are methods of treating a lysosomal storage disorder in a mammal which method includes administering AAV particles encoding a polypeptide to the central nervous system of the mammal. AAV particles may be delivered by direct injection into the brain, spinal cord, cerebral spinal fluid or a portion thereof for expression.

### RELATED APPLICATIONS

This application claims priority to U.S. Provisional Patent Application No. 64/418,033, filed Nov. 4, 2016. The entire contents of the foregoing application is incorporated herein by reference, including all text, tables, sequence listing and drawings.

### INTRODUCTION

Gene transfer is now widely recognized as a powerful tool for analysis of biological events and disease processes at both the cellular and molecular level. More recently, the application of gene therapy for the treatment of human diseases, either inherited (e.g., ADA deficiency) or acquired (e.g., cancer or infectious disease), has received considerable attention.

Traditionally, gene therapy has been defined as a procedure in which a therapeutic gene is introduced into cells of a mammal in order to correct an inborn genetic error. Although more than 4500 human diseases are currently classified as genetic, specific mutations in the human genome have been identified for relatively few of these diseases. Until recently, these rare genetic diseases represented the exclusive targets of gene therapy efforts. Accordingly, most of the NIH approved gene therapy protocols to date have been directed toward the introduction of a functional copy of a defective gene into the somatic cells of an individual having a known inborn genetic error. Only recently, have researchers and clinicians begun to appreciate that most human cancers, certain forms of cardiovascular disease, and many degenerative diseases also have important genetic components, and for the purposes of designing novel gene therapies, should be considered “genetic disorders.” Therefore, gene therapy has more recently been broadly defined as the correction of a disease phenotype through the introduction of new genetic information into the affected organism.

In in vivo gene therapy, a transferred gene is introduced into cells of the recipient organism in situ that is, within the recipient. In vivo gene therapy has been examined in several animal models. Several recent publications have reported the feasibility of direct gene transfer in situ into organs and tissues such as muscle, hematopoietic stem cells, the arterial wall, the nervous system, and lung. Direct injection of DNA into skeletal muscle, heart muscle and injection of DNA-lipid complexes into the vasculature also has been reported to yield a detectable expression level of the inserted gene product(s) in vivo.

Treatment of diseases of the central nervous system, e.g., inherited genetic diseases of the brain, remains an intractable problem. Examples of such are the lysosomal storage diseases and Alzheimer's disease. Collectively, the incidence of lysosomal storage diseases (LSD) is 1 in 10,000 births worldwide, and in 65% of cases, there is significant central nervous system (CNS) involvement. Proteins deficient in these disorders, when delivered intravenously, do not cross the blood-brain barrier, or, when delivered directly to the brain, are not widely distributed. Thus, therapies for the CNS deficits need to be developed.

### SUMMARY

The invention provides methods and uses of treating a primate having a lysosomal storage disease (LSD). In one embodiment, a method or use includes providing AAV particles comprising an AAV capsid protein; a nucleic acid inserted between a pair of AAV inverted terminal repeats (ITRs), the nucleic acid encoding a polypeptide having lysosomal hydrolase activity; and an expression control element driving expression of said nucleic acid; wherein the AAV particles are capable of transducing cells of said mammal and providing expression of said polypeptide; and administering or delivering the AAV particles to the CNS of the mammal.

In one embodiment, the polypeptide has tripeptidyl-peptidase 1 (TPP1) activity. In other embodiments, the polypeptide comprises TPP1, a pro-enzyme thereof, or an enzymatically active variant thereof. In further embodiments, the nucleic acid encodes a protein with TPP1 activity and having 80% or more identity to human TPP1 set forth as SEQ ID NO:1. In still further embodiments, the nucleic acid encodes mammalian (e.g., human) TPP1.

In additional embodiments, one or more of the AAV ITRs comprise one or more AAV2 ITRs.

In certain embodiments, an expression control element driving expression of said nucleic acid comprises a CMV enhancer.

In certain embodiments, an expression control element driving expression of said nucleic acid comprises a beta actin promoter.

In certain embodiments, an expression control element driving expression of said nucleic acid comprises a chicken beta actin promoter.

In certain embodiments, an expression control element driving expression of said nucleic acid comprises a CMV enhancer and a chicken beta actin promoter.

In certain embodiments, an expression control element driving expression of said nucleic acid comprises a sequence having 80% or more identity to CMV enhancer set forth in SEQ ID NO:3 and/or a sequence having 80% or more identity to chicken beta actin promoter set forth in SEQ ID NO:3.

In certain embodiments, an expression control element driving expression of said nucleic acid comprises a sequence having 80% or more identity to SEQ ID NO:3.

In certain embodiments, an expression control element driving expression of said nucleic acid comprises SEQ ID NO:3.

### DESCRIPTION OF THE DRAWINGS

FIG. 1A-1H shows TPP1 enzyme levels for the Dosing Study in brain parenchyma, CSF and peripheral tissues. Recombinant TPP1 enzyme after AAV4CAGhTPP1 injection in CLN2^{-/-} mice compared to endogenous levels (red line) of CLN2^{+/+} animals. (Statistical analyses: Not all of the groups passed normality test. Non-parametric analysis with Krukal-Wallis test followed by Dunn's multiple comparisons test were performed. *P<0.05, **P<0.01, ****P<0.0001)

FIG. 2A shows comparative TPP1 expression in cerebrospinal fluid (CSF) after rostral or caudal injection.

FIG. 2B shows TPP1 expression in striatum after rostral or caudal injection.

FIG. 2C shows TPP1 expression in thalamus after rostral or caudal injection.

FIG. 2D shows TPP1 expression in medulla oblongata after rostral or caudal injection.

FIG. 2E shows TPP1 expression in cerebellum after rostral or caudal injection.

FIG. 2F shows TPP1 expression in occipital cortex after rostral or caudal injection.

FIG. 2G shows TPP1 expression in prefrontal cortex after rostral or caudal injection.

FIG. 3A-3J shows AAV4.CAGhTPP1 genome copies for the Dosing Study in CLN2^{-/-} brain and peripheral tissues 5 weeks post-injection at three doses.

FIG. 4A-4B shows tremor phenotype quantification for the Dosing Study in 12 week old CLN2^{-/-} mice 5 weeks after AAV4.CAGhTPP1 injection at three doses. A) Spectrum pattern of the tremor amplitude in deciBeltVolts (dBV) at different tremor frequencies in hertzs (Hz). (B) Area under the curve of the tremor spectrum (from panel A) followed by one-way ANOVA with Tukey's multiple comparisons test were performed. *p<0.05, ***p<0.001)

FIG. 5A-FIG. 5G shows recombinant TPP1 enzyme activity from the Stability Study after 5e10 vg AAV4.CAGhTPP1 injection in CLN2^{-/-} mice.

FIG. 6 shows TPP1 levels (pmol TPP1/mg protein) in CSF of non-human primates after unilateral injection of AAV2 vector. CAGhTPP1 in brain ventricle. Data represents TPP1 levels over time. Day 0 represents TPP1 endogenous levels before injection. N=3 animals.

### DETAILED DESCRIPTION

Provided herein are methods and uses for administering to a mammal, in need of a method described herein, that is suspected of having or that has a lysosomal storage disease (LSD). In certain embodiments, a method or use described herein is used to treat, prevent, inhibit, reduce, decrease or delay the number, severity, frequency, progression or onset of one or more symptoms of an LSD.

Non-limiting examples of LSDs include Infantile Lipofuscinosis or Late infantile Neuronal Ceroid Lipofuscinosis (LINCL), Gaucher, Juvenile Batten, Fabry, MLD, Sanfilippo A, Late Infantile Batten, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type II/III, or Sandhoff disease.

LSDs are often caused by a genetic abnormality (e.g., mutation, deletion, insertion) in the gene encoding a tripeptidyl peptidase-1 (TPP1) enzyme thereby leading to a deficiency of functional TPP1 enzyme activity. In humans, TPP1 is encoded by the CLN2 gene, sometimes called the TPP1 gene (see, e.g., SEQ ID NO:2). For example, Late infantile Neuronal Ceroid Lipofuscinosis (LINCL) is a childhood neurodegenerative disease caused most often by deficiency of TPP1 activity, due to mutations in CLN2. Development is normal up to ages 2-4 years after which manifestations of LINCL present as motor and mental decline, seizure disorder and visual deficits. Death generally occurs within the first decade of life. Most cases of LINCL are due to mutations in CLN2, which induce a deficiency of the soluble lysosomal enzyme tripeptidyl peptidase-1 (TPP1). TPP1 is synthetized as a mannose-6-phophate pro-enzyme and, similar to other soluble lysosomal hydrolases, the pro-enzyme is largely targeted to the lysosome but can also be released from the cell via the secretory pathway. As such, cellular uptake by the same or neighboring cells, and subsequent lysosomal delivery and activation of the proenzyme to the active form, can occur.

In certain embodiments, provided herein are methods of treating a mammal having, or suspected of having an LSD by administering, directly to a tissue or fluid of the central nervous system, AAV particles that direct the expression of polypeptide having TPP1 activity (referred to herein as AAV-TPP1 particles). Disclosed herein are data showing AAV delivery/administration to the brain and/or spinal cord in an animal model of a lysosomal storage disorder.

In certain embodiments, AAV-TPP1 particles are administered to the cisternae magna, intraventricular space, brain ventricle, subarachnoid space, intrathecal space and/or ependyma of said mammal. In additional embodiments, AAV-TPP1 particles are administered to the cerebral spinal fluid (CSF) of said mammal. In further embodiments, AAV-TPP1 particles are administered to the ventricular system.). In still further embodiments, AAV-TPP1 particles are administered to the rostral lateral ventricle; and/or administered to the caudal lateral ventricle; and/or administered to the right lateral ventricle; and/or administered to the left lateral ventricle; and/or administered to the right rostral lateral ventricle; and/or administered to the left rostral lateral ventricle; and/or administered to the right caudal lateral ventricle; and/or administered to the left caudal lateral ventricle.

In still additional embodiments, AAV-TPP1 particles are administered such that the AAV particles contact ependymal cells of said mammal. Such ependymal express the encoded polypeptide and optionally the polypeptide is expressed by the cells. In particular embodiments, the polypeptide is expressed and/or is distributed in the lateral ventricle, CSF, brain (e.g., striatum, thalamus, medulla, cerebellum, occipital cortex, and/or prefrontal cortex), and/or CNS.

Any suitable mammal can be treated by a method or use described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models used for the study of LSDs, such as LINCL.

Subjects treated by a method or composition described herein include adults (18 years or older) and children (less than 18 years of age). Children range in age from 1-2 years old, or from 2-4, 4-6, 6-18, 8-10, 10-12, 12-15 and 15-18 years old. Children also include infants. Infants typically range from 1-12 months of age.

Adeno associated virus (AAV) is a small nonpathogenic virus of the parvoviridae family. To date, numerous serologically distinct AAVs have been identified, and more than a dozen have been isolated from humans or primates. AAV is distinct from the other members of this family by its dependence upon a helper virus for replication.

AAV genomes been shown to stably integrate into host cellular genomes; possess a broad host range; transduce both dividing and non-dividing cells in vitro and in vivo and maintain high levels of expression of the transduced genes. AAV viral particles are heat stable, resistant to solvents, detergents, changes in pH, temperature, and can be concentrated on CsCl gradients or by other means. The AAV genome comprises a single-stranded deoxyribonucleic acid (ssDNA), either positive- or negative-sensed. In the absence of a helper virus, AAV may integrate in a locus specific manner, for example into the q arm of chromosome 19. The approximately 5 kb genome of AAV consists of one segment of single stranded DNA of either plus or minus polarity. The ends of the genome are short inverted terminal repeats which can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsulated into virus (e.g., AAV) particles. Thus, a vector “genome” refers to nucleic acid that is packaged or encapsulated by virus (e.g., AAV).

The AAV virion (particle) is a non-enveloped, icosahedral particle approximately 25 nm in diameter. The AAV particle comprises a capsid of icosahedral symmetry comprised of three related capsid proteins, VP1, VP2 and VP3, which interact together to form the capsid. The right ORF often encodes the capsid proteins VP1, VP2, and VP3. These proteins are often found in a ratio of 1:1:10 respectively, but may be in varied ratios, and are all derived from the right-hand ORF. The capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles. An AAV particle is a viral particle comprising an AAV capsid. In certain embodiments the genome of an AAV particle encodes one, two or all VP1, VP2 and VP3 polypeptides.

The genome of most native AAVs often contain two open reading frames (ORFs), sometimes referred to as a left ORF and a right ORF. The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank its single stranded viral DNA genome. The ITR sequences often comprise about 145 bases each. Within the ITR region, two elements have been described which are thought to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

In certain embodiments an AAV (e.g., an rAAV) comprises two ITRs. In certain embodiments an AAV (e.g., an rAAV) comprises a pair of ITRs. In certain embodiments an AAV (e.g., an rAAV) comprises a pair of ITRs that flank (i.e., are at each 5′ and 3′ end) of a polynucleotide that at least encodes a polypeptide having TPP1 enzyme activity.

The term “vector” refers to small carrier nucleic acid molecule, a plasmid, virus (e.g., AAV vector), or other vehicle that can be manipulated by insertion or incorporation of a nucleic acid. Vectors such as AAV can be used to introduce/transfer polynucleotides into cells, such that the polynucleotide therein is transcribed and subsequently translated by the cells.

An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, ITR(s), polyadenylation signal.

A viral vector is derived from or based upon one or more nucleic acid elements that comprise a viral genome. Particular viral vectors include adeno-associated virus (AAV) vectors. Also provided are vectors (e.g., AAV) comprising a nucleic acid sequence encoding a TPP1 polypeptide, variant or subsequence (e.g., a polypeptide fragment having TPP1 enzyme activity).

The term “recombinant,” as a modifier of vector, such as recombinant viral, e.g., lenti- or parvo-virus (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. An example of a recombinant polynucleotide would be where a nucleic acid (e.g., gene) encoding a TPP1 polypeptide is cloned into a vector, with or without 5′, 3′ and/or intron regions that the gene is normally associated within the viral (e.g., AAV) genome. Although the term “recombinant” is not always used herein in reference to vectors, such as viral and AAV vectors, as well as sequences such as polynucleotides, recombinant forms including polynucleotides, are expressly included in spite of any such omission.

A recombinant viral “vector” or “AAV vector” is derived from the wild type genome of a virus, such as AAV by using molecular methods to remove the wild type genome from the virus (e.g., AAV), and replacing with a non-native nucleic acid, such as a TPP1 encoding nucleic acid sequence. Typically, for AAV one or both inverted terminal repeat (ITR) sequences of AAV genome are retained in the AAV vector. A “recombinant” viral vector (e.g., rAAV) is distinguished from a viral (e.g., AAV) genome, since all or a part of the viral genome has been replaced with a non-native sequence with respect to the viral (e.g., AAV) genomic nucleic acid such as TPP1 encoding nucleic acid sequence. Incorporation of a non-native sequence therefore defines the viral vector (e.g., AAV) as a “recombinant” vector, which in the case of AAV can be referred to as a “rAAV vector.”

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is an rAAV particle. A rAAV particle often comprises an AAV vector, or a portion thereof. A rAAV particle can be one or more AAV particles (e.g., a plurality of AAV particles). rAAV particles typically comprise proteins that encapsulate or package the rAAV vector genome (e.g., capsid proteins).

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 or AAV-2i8. In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV particle excludes certain serotypes. In one embodiment, a rAAV particle is not an AAV4 particle. In certain embodiments, a rAAV particle is antigenically or immunologically distinct from AAV4. Distinctness can be determined by standard methods. For example, ELISA and Western blots can be used to determine whether a viral particle is antigenically or immunologically distinct from AAV4. Furthermore, in certain embodiments a rAAV2 particle retains tissue tropism distinct from AAV4.

In certain embodiments, a rAAV vector based upon a first serotype genome is identical to the serotype of one or more of the capsid proteins that package the vector. In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., a AAV1, AAV3, AAV4, AAV5, AAV6, AAV7, AAV8, AAV9, AAV10, AAV11, AAV12, Rh10, Rh74 or AAV-2i8 serotype or variant thereof.

Recombinant AAV vectors that include a polynucleotide that directs the expression of a polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact AAV particle that is transduction-competent is configured to transduce a mammalian cell. An AAV particle configured to transduce a mammalian cell is often not replication competent, and requires additional protein machinery to self-replicate. Thus an AAV particle that is configured to transduce a mammalian cell is engineered to bind and enter a mammalian cell and deliver a nucleic acid to the cell, wherein the nucleic acid for delivery is often positioned between a pair of AAV ITRs in the AAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, 293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

In certain embodiments, an AAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, an AAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use of an AAV2 particle. In a particular aspect, an AAV2 particle is a recombinant AAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV2 particle (e.g., a capsid of an AAV2 particle) comprises a VP1 polypeptide having at least 60%, at least 70% identity, at least 75% identity, at least 80% identity, at least 85% identity, at least at least 90% identity, at least 95% identity, at least 98% identity, at least 99% identity, or even 100% identity to wild-type AAV2 VP1 capsid. In certain embodiments an AAV2 particle comprises a VP1 polypeptide that is about 63% or more identical (e.g., 63% identity) to the polypeptide having the amino acid sequence of AAV2 VP1 capsid protein. AAV2 capsid sequence and AAV4 capsid sequence are about 60% identical. In certain embodiments, the AAV2 VP1 capsid protein has a sequence that has at least 65% identity to wild-type AAV2 VP1 capsid. In certain embodiments, the AAV2 VP1 capsid protein comprises wild-type AAV2 VP1 capsid.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Any suitable length of DNA can be incorporated into an AAV particle. Suitable DNA molecules for use in rAAV vectors can about 5 kilobases (kb), less than about 5 kb, less than about 4.5 kb, less than about 4 kb, less than about 3.5 kb, less than about 3 kb, or less than about 2.5 kb.

A “transgene” is used herein to conveniently refer to a nucleic acid that is intended or has been introduced into a cell or organism. Transgenes include any nucleic acid, such as a gene that encodes a polypeptide or protein (e.g., TPP1), and are generally heterologous with respect to naturally occurring AAV genomic sequences.

In a cell having a transgene, the transgene is often introduced/transferred by way of a vector, such as a rAAV particle. Introduction of a transgene into a cell by a rAAV particle is often referred to as “transduction” of the cell. The term “transduce” refers to introduction of a molecule such as a nucleic acid into a cell or host organism by way of a vector (e.g., an AAV particle). The transgene may or may not be integrated into genomic nucleic acid of a transduced cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extra chromosomally, or only transiently. A “transduced cell” is a cell into which the transgene has been introduced by way of transduction. Thus, a “transduced” cell is a cell into which, or a progeny thereof in which a nucleic acid has been introduced. A transduced cell can be propagated and the introduced protein expressed, or nucleic acid transcribed. For gene therapy uses and methods, a transduced cell can be in a mammal.

TPP1 is a lysosomal serine protease encoded by the CLN2 gene (TPP1 gene). The amino acid sequence of human TPP1 is set forth as SEQ ID NO:1. The nucleic acid sequence of human TPP1 is set forth as SEQ ID NO:2. Human TPP1 comprises tripeptidyl-peptidase I activity (TPP1 enzyme activity). TPP1 activity comprises a non-specific lysosomal peptidase activity which generates tripeptides from the breakdown products produced by lysosomal proteinases. Substrate-specificity studies indicate that TPP1 primarily cleaves tripeptides from unsubstituted amino termini in peptides and proteins. Endogenously expressed TPP1 is synthesized as a catalytically-inactive enzyme. After targeting into lysosomes, because of the acidic environment, the TPP1 is auto-catalytically processed into a mature active enzyme. The activity of TPP1 can be measured and/or quantitated in vitro using known methods. See, for example, Junaid et al., 1999.

A polypeptide comprising TPP1 activity refers to a TPP1 protein of a mammal, or a portion thereof, that displays at least 50%, at least 60%, at least 70%, at least 75%, at least 80%, at least 85%, at least 90%, at least 95%, or about 100% of the peptidase activity of the human TPP1 of SEQ ID NO:1 as assayed using a suitable peptide substrate, for example, as assayed by the method of Junaid et al., 1999 or another comparable method. In certain embodiments a polypeptide comprising TPP1 activity refers to a TPP1 protein of a mammal, or a subsequence or variant thereof, that displays at least at least 50%, at least 60%, at least 70%, at least 75%, at least 80%, at least 85%, at least 90%, at least 95%, or about 100% of the peptidase activity of the human TPP1 of SEQ ID NO:1.

A polypeptide comprising TPP1 activity may comprise a truncated, mutated, chimeric, or modified form of a TPP1 polypeptide that retains at least partial TPP1 activity. A polypeptide comprising TPP1 activity may comprise a TPP1 protein, or a portion thereof, obtained from any suitable organism (e.g., from a mammal, from a human, from a non-human mammal, e.g., from a dog, pig, cow, or the like). In certain embodiments a polypeptide comprising TPP1 activity has at least 60% identity, at least 70% identity, at least 75% identity, at least 80% identity, at least 85% identity, at least 90% identity, at least 95% identity, at least 98% identity, or 100% identity to the TPP1 protein set forth in SEQ ID NO:1.

In certain embodiments a rAAV particle comprises an AAV capsid protein and a nucleic acid encoding a polypeptide comprising TPP1 activity. In certain embodiments a rAAV particle comprises an AAV capsid protein and a nucleic acid that directs the expression and/or secretion of a polypeptide comprising TPP1 activity.

In certain embodiments a rAAV particle comprises an AAV capsid protein and a nucleic acid encoding a TPP1 polypeptide, or enzymatically active portion thereof. In certain embodiments a rAAV particle comprises an AAV capsid protein and a nucleic acid that directs the expression and/or secretion of a TPP1 polypeptide, or enzymatically active portion thereof. In certain embodiments, a nucleic acid being administered encodes TPP1, a TPP1 that has substantial identity to wild type TPP1, and/or a variant, mutant or fragment of a TPP1. In certain embodiments a TPP1 polypeptide has at least 60% identity, at least 70% identity, at least 75% identity, at least 80% identity, at least 85% identity, at least 90% identity, at least 95% identity, at least 98% identity, or 100% identity to the protein set forth in SEQ ID NO:1.

In certain embodiments a rAAV particle comprises a nucleic acid having at least 50% identity, at least 60% identity, at least 70% identity, at least 75% identity, at least 80% identity, at least 85% identity, at least 90% identity, at least 95% identity, at least 98% identity, or 100% identity to the nucleic acid set forth in SEQ ID NO:2. In certain embodiments a nucleic acid encoding a TPP1 activity or encoding or directing the expression of a TPP1 polypeptide is a nucleic acid having at least 50% identity, at least 60% identity, at least 70% identity, at least 75% identity, at least 80% identity, at least 85% identity, at least 90% identity, at least 95% identity, at least 98% identity, or 100% identity to the nucleic acid set forth in SEQ ID NO:2.

A representative human TPP1 amino acid sequence is depicted in SEQ ID NO: 1. A representative human TPP1 nucleic acid sequence is depicted in SEQ ID NO:2.

In certain embodiments a method or use includes administering or delivering AAV-TPP1 particles to a mammal and optionally administering one or more immunosuppressive agents to the mammal. In certain embodiments a method or use includes administering or delivering AAV-TPP1 particles to a mammal and optionally administering 2, 3, 4 or more immunosuppressive agents to the mammal. In certain embodiments a method or use includes administering or delivering AAV-TPP1 particles to a mammal and optionally administering two immunosuppressive agents to the mammal. In one representative embodiment, a method or use of treating a mammal includes administering or delivering AAV-TPP1 particles to a mammal and administering first and second immunosuppressive agents to the mammal.

Where two or more immunosuppressive agents are administered, each immunosuppressive agent is distinct and/or different (e.g., each agent differs in structure and/or mechanism of action). In certain embodiments, an immunosuppressive agent is an anti-inflammatory agent. In certain embodiments, an immunosuppressive agent is mycophenolate, or a derivative thereof. An example of such a mycophenolate derivative is mycophenolate mofetil (MMF). In certain embodiments, an immunosuppressive agent is cyclosporine or a derivative thereof. In certain embodiments a first immunosuppressive agent comprises cyclosporine and a second immunosuppressive agent comprises mycophenolate, or a derivative thereof (e.g., MMF). In certain embodiments a first immunosuppressive agent comprises cyclosporine and a second immunosuppressive agent comprises MMF.

In certain embodiments, an immunosuppressive agent is administered before, during and/or after administration of AAV-TPP1 particles to a mammal. In certain embodiments, an immunosuppressive agent is administered concurrently with administration of AAV-TPP1 particles to a mammal. In certain embodiments, an immunosuppressive agent is administered after administration of AAV-TPP1 particles to a mammal.

In certain embodiments, a first immunosuppressive agent is administered to a mammal at least about 1 to about 7 days before, or about 1, about 2, about 3, about 4 or about 5 weeks before administration of AAV-TPP1 particles to a mammal and a second immunosuppressive agent is administered about 1 to about 7 days before, about 1, about 2, about 3, about 4 or about 5 weeks before, during and/or within about 10, about 20, about 30, about 40, about 50, about 100, about 200, about 300, about 350, about 400 or about 500 days after administration of AAV-TPP1 particles to the mammal. In certain embodiments, cyclosporine is administered to a mammal at least about 1 to about 7 days before, or about 1, about 2, about 3, about 4 or about 5 weeks before administration of AAV-TPP1 particles to a mammal, and mycophenolate or a derivative thereof (e.g., MMF) is administered about 1 to about 7 days before, about 1, about 2, about 3, about 4 or about 5 weeks before, during and/or within about 10, about 20, about 30, about 40, about 50, about 100, about 200, about 300, about 350, about 400 or about 500 days after administration of AAV-TPP1 particles to the mammal. In certain embodiments, cyclosporine is administered about 1 to about 7 days before, or about 1, about 2, about 3, about 4 or about 5 weeks before administration of AAV-TPP1 particles and at regular intervals after treatment, and mycophenolate or a derivative thereof (e.g., MMF) is administered once at about 1 to about 7 days before, about 1, about 2, about 3, about 4 or about 5 weeks before, during and/or within about 10 to about 40 days after administration of AAV-TPP1 particles to the mammal.

An immunosuppressive agent can be administered at any suitable dose. In certain embodiments, cyclosporine is administered at a dosage of about 1 to about 50 mg/kg, about 1 to about 20 mg/kg, or about 5 to about 10 mg/kg at a frequency of once, twice or three times a day, to once every other day. In certain embodiments cyclosporine is administered at about 10 mg/kg twice a day. In certain embodiments, cyclosporine is administered at about 10 mg/kg twice a day for a period of at least about 1, about 2, about 3, about 4 or about 5 months. In certain embodiments, a dosage of cyclosporine is tapered down to a dose of less than about 5 mg/kg, or less than about 2 mg/kg about 1 to about 2 months after administration or use of AAV-TPP1 particles to a mammal.

In certain embodiments, mycophenolate or a derivative thereof (e.g., MMF), is administered at a dosage of about 1 to about 100 mg/kg, about 1 to about 50 mg/kg, about 1 to about 25 mg/kg, or about 5 to about 20 mg/kg at a frequency of once, twice or three times a day, to once every other day. In certain embodiments, mycophenolate or a derivative thereof (e.g., MMF) is administered at about 10 to about 20 mg/kg once a day. In certain embodiments, a dosage of mycophenolate or a derivative thereof (e.g., MMF) is reduced down to a dose of less than about 5 mg/kg, or less than about 2 mg/kg about 1 to about 2 months after the administration of AAV-TPP1 particles to a mammal.

A rAAV particle and/or immunosuppressive agent can be formulated in any suitable formulation suitable for a particular route of administration. Various pharmaceutically acceptable formulations are commercially available and obtainable by a medical practitioner.

A rAAV particle can be administered by any suitable route. In certain embodiments a method or use includes administering AAV-TPP1 particles to the central nervous system (CNS) of a mammal (e.g., a mammal having a LSD). In certain embodiments, the central nervous system includes brain, spinal cord and cerebral spinal fluid (CSF). In certain embodiments, a method or use includes administering AAV-TPP1 particles to the brain or spinal cord or CSF of a mammal. In certain embodiments, AAV-TPP1 particles are administered to a portion of brain or spinal cord.

In certain embodiments, AAV-TPP1 particles are administered to one or more of cisterna magna, intraventricular space, brain ventricle, subarachnoid space, intrathecal space and/or ependyma of said mammal. In certain embodiments, AAV-TPP1 particles are administered to the cerebral spinal fluid (CSF) of said mammal. In further embodiments, AAV-TPP1 particles are administered to the ventricular system. In still further embodiments, AAV-TPP1 particles are administered to one or more of the rostral lateral ventricle, the caudal lateral ventricle, the right lateral ventricle, the left lateral ventricle, the right rostral lateral ventricle, the left rostral lateral ventricle, the right caudal lateral ventricle and/or the left caudal lateral ventricle.

An immunosuppressive agent can be administered by any suitable route. In certain embodiments, an immunosuppressive agent is administered orally. In certain embodiments, mycophenolate or a derivative thereof, such as Mycophenolate Mofetil (MMF), is administered orally. In certain embodiments, cyclosporine is administered orally. An immunosuppressive agent can also be administered parenterally (e.g., intramuscularly, intravenously, subcutaneously), or administered by injection to the brain, spinal cord, or a portion thereof (e.g., injected into the CSF).

In certain embodiments, a composition including AAV-TPP1 particles, and optionally an immunosuppressive agent, are administered to one or more of a mammal's cisterna magna and/or the mammal's brain ventricle, subarachnoid space, and/or intrathecal space, and/or ependyma. For example, AAV-TPP1 particles can be delivered directly to the cisterna magna, intraventricular space, a brain ventricle, subarachnoid space, intrathecal space and/or ependyma. In certain embodiments, a method or use includes administering AAV-TPP1 particles to the ependyma of a mammal.

In certain embodiments AAV-TPP1 particles are administered to one or more cells that contact the CSF in a mammal, for example by contacting cells with AAV-TPP1 particles. Non-limiting examples of cells that contact the CSF include ependymal cells, pial cells, endothelial cells and/or meningeal cells. In certain embodiments AAV-TPP1 particles are administered to ependymal cells. In certain embodiments AAV-TPP1 particles are delivered to ependymal cells, for example by contacting ependymal cells with AAV-TPP1 particles.

In certain embodiments, AAV-TPP1 particles are delivered locally. “Local delivery” refers to delivery of an active agent directly to a target site within a mammal (e.g., directly to a tissue or fluid). For example, an agent can be locally delivered by direct injection into an organ, tissue or specified anatomical location. In certain embodiments, AAV-TPP1 particles are delivered or administered by direct injection to the brain, spinal cord, or a tissue or fluid thereof (e.g., CSF, such as ependymal cells, pial cells, endothelial cells and/or meningeal cells). For example AAV-TPP1 particles can be directly delivered, by way of direct injection, to the CSF, cisterna magna, intraventricular space, a brain ventricle, subarachnoid space and/or intrathecal space and/or ependyma. In certain embodiments, AAV-TPP1 particles are delivered to a tissue, fluid or cell of the brain or spinal cord by direct injection into a tissue or fluid of the brain or spinal cord. In certain embodiments, AAV-TPP1 particles are not delivered systemically by, for example, intravenous, subcutaneous, or intramuscular injection, or by intravenous infusion. In certain embodiments, AAV-TPP1 particles are delivered to a tissue or fluid of the brain or spinal cord by stereotactic injection.

In certain embodiments one or more AAV-TPP1 particles are delivered or administered by direct injection of AAV-TPP1 particles to the brain, spinal cord, or a tissue or fluid thereof (e.g., CSF such as ependyma). In a particular aspect, AAV-TPP1 particles transduce ependymal cells, pial cells, endothelial cells and/or meningeal cells.

An effective amount of rAAV particles, such as AAV-TPP1 particles, can be empirically determined. Administration can be effected in one or more doses, continuously or intermittently throughout the course of treatment. Effective doses of administration can be determined by those of skill in the art and may vary according to the AAV serotype, viral titer and the weight, condition and species of mammal being treated. Single and multiple administrations can be carried out with the dose level, target and timing being selected by the treating physician. Multiple doses may be administered as is required to maintain adequate enzyme activity, for example.

In certain embodiments, a plurality of AAV-TPP1 particles are administered. As used herein, a plurality of AAV particles refers to about 1×10⁵ to about 1×10¹⁸ particles.

In certain embodiments, rAAV particles, such as AAV-TPP1 particles, are administered at a dose of about 1×10⁵ to about 1×10¹⁶ vg/ml in about 1 to about 5 ml; at a dose of about 1 to about 3 ml of 1×10⁷ to about 1×10¹⁴ vg/ml; or at a dose of about 1 to about 2 ml of 1×10⁸ to about 1×10¹³ vg/ml. In certain embodiments, rAAV particles, such as AAV-TPP1 particles, are administered at a dose of about 1×10⁸ to about 1×10¹⁵ vg/kg body weight of the mammal being treated. For example, rAAV particles, such as AAV-TPP1 particles, can be administered at a dose of about 1×10⁸ vg/kg, about 5×10⁸ vg/kg, about 1×10⁹ vg/kg, about 5×10⁹ vg/kg, about 1×10¹⁰ vg/kg, about 5×10¹⁰ vg/kg, about 1×10¹¹ vg/kg, about 5×10¹¹ vg/kg, about 1×10¹² vg/kg, about 5×10¹² vg/kg, about 1×10¹³ vg/kg, about 5×10¹³ vg/kg, about 1×10¹⁴ vg/kg, about 5×10¹⁴ vg/kg, or about 1×10¹⁵ vg/kg body weight of the mammal being treated.

Pharmaceutical forms suitable for injection or infusion of rAAV particles, such as AAV-TPP1 particles, can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of rAAV particles, such as AAV-TPP1 particles, can optionally include the following components: a sterile diluent such as water for injection, saline solution, such as phosphate buffered saline (PBS), artificial CSF, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

rAAV particles, such as AAV-TPP1 particles, and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the amount of rAAV particles (e.g., AAV-TPP1 particles) believed necessary to produce the desired effect(s). The amount necessary can be formulated in a single dose, or can be formulated in multiple dosage units. The dose may be adjusted to a suitable rAAV particles concentration, optionally combined with an anti-inflammatory agent, and packaged for use.

In one embodiment, pharmaceutical compositions will include sufficient genetic material (rAAV particles) to provide a therapeutically effective amount, i.e., an amount sufficient to reduce or ameliorate symptoms of a disease state in question or an amount sufficient to confer the desired benefit. Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences, 1991.

Formulations containing rAAV particles, such as AAV-TPP1 particles, will contain an effective amount of the rAAV particles in a vehicle, the effective amount being readily determined by one skilled in the art. The rAAV particles, such as AAV-TPP1 particles, may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

In certain embodiments a method includes administering a plurality of rAAV particles, such as AAV-TPP1 particles, to a mammal (e.g., a mammal having an LSD such as LINCL) as set forth herein, where severity, frequency, progression or time of onset of one or more symptoms of a LSD are decreased, reduced, prevented, inhibited or delayed. The term “time of onset” refers to a point in time after a first administration of AAV-TPP1 particles that a symptom of LSD is first observed or detected. Non-limiting symptoms of LSD in which severity, frequency, progression or time of onset of one or more symptoms of a LSD are decreased, reduced, prevented, inhibited or delayed include a proprioceptive response, nystagmus, menace, pupillary light reflex, cerebellar ataxia and intention tremor. The severity, frequency, progression or time of onset of one or more symptoms of a LSD can be subjectively determined by a standardized clinical neurologic examination (e.g., see Lorenz et al., 2011).

A delay in the time of onset of a symptom associated with LSD can be determined by comparing the time of onset of a symptom for a mammal treated with AAV-TPP1 particles to one or more mammals treated without AAV-TPP1 particles. In certain embodiments a method includes administering a plurality of AAV-TPP1 particles to the central nervous system, or portion thereof, of a mammal (e.g., a mammal having an LSD) and severity, frequency, progression or time of onset of one or more symptoms of a LSD are decreased, reduced, prevented, inhibited or delayed by at least about 5 to about 10, about 10 to about 25, about 25 to about 50, or about 50 to about 100 days.

In certain embodiments, a method or use includes administering rAAV particles to the brain or spinal cord, or portion thereof, of a mammal where the rAAV particles are configured to transduce cells of the mammal and direct expression of a polypeptide having TPP1 activity in the mammal. In certain embodiments, the polypeptide is expressed and/or detected in one or more peripheral organs (e.g., in spleen and/or heart).

In certain embodiments, a method or use includes administering rAAV particles to the brain or spinal cord, or portion thereof, of a mammal where the rAAV particles are configured to transduce brain or spinal cord cells of the mammal and direct expression of the polypeptide having TPP1 activity in the brain or spinal cord of the mammal. In certain embodiments, the polypeptide is expressed and/or detected in a central nervous tissue (e.g., brain, e.g., striatum, thalamus, medulla, cerebellum, occipital cortex, prefrontal cortex) distal to the administration site. In certain embodiments, the polypeptide is present or detected broadly in a central nervous tissue (e.g., brain, e.g., striatum, thalamus, medulla, cerebellum, occipital cortex, and/or prefrontal cortex) that reflects distribution away from the administration site and optionally throughout a central nervous tissue (e.g., brain, e.g., striatum, thalamus, medulla, cerebellum, occipital cortex, and/or prefrontal cortex).

The terms “polynucleotide” and “nucleic acid” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism. Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

A nucleic acid is “operably linked” when it is placed into a functional relationship with another nucleic acid sequence. A nucleic acid encoding a polypeptide, or a nucleic acid directing expression of a TPP1 polypeptide (e.g., a polypeptide having TPP1 activity) may include an inducible promoter, or a tissue-specific promoter for controlling transcription of the encoded polypeptide.

In certain embodiments, CNS-specific or inducible promoters, enhancers and the like, are employed in the methods described herein. Non-limiting examples of CNS-specific promoters include those isolated from the genes from myelin basic protein (MBP), glial fibrillary acid protein (GFAP), and neuron specific enolase (NSE). Non-limiting examples of inducible promoters include DNA responsive elements for ecdysone, tetracycline, hypoxia and IFN.

In certain embodiments, an expression control element comprises a CMV enhancer. In certain embodiments, an expression control element comprises a beta actin promoter. In certain embodiments, an expression control element comprises a chicken beta actin promoter. In certain embodiments, an expression control element comprises a CMV enhancer and a chicken beta actin promoter.

In certain embodiments, an expression control element comprises a sequence having 80% or more identity to CMV enhancer set forth in SEQ ID NO:3 and/or a sequence having 80% or more identity to chicken beta actin promoter set forth in SEQ ID NO:3. In certain embodiments, an expression control element comprises a sequence having 80% or more identity to SEQ ID NO:3. In certain embodiments, an expression control element comprises SEQ ID NO:3.

A polypeptide can be targeted for delivery to an extracellular, intracellular or membrane location. A gene product secreted from cells typically has a secretion “signal” for secretion from the cell to the extracellular milieu. An expression vector can also be constructed to include a secretion “signal.” A gene product may also be retained within the cell. In a similar manner, a gene product may include or the expression vector can be constructed to include “retention” signal sequences for anchoring the polypeptide within the cell plasma membrane. For example, membrane proteins have hydrophobic transmembrane regions, which maintain protein in the membrane.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation in TPP1.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein, e.g., a variant TPP1 protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a TPP1 protein encoded thereby, and some codons of the nucleic acid variant will be changed which in turn changes the amino acids of a TPP1 protein encoded thereby.

The term “polypeptide” as used herein refers to a polymer of amino acids and includes full-length proteins and fragments thereof. Thus, “protein” and “polypeptide” are often used interchangeably herein. The “polypeptides” encoded by a “nucleic acid” or “polynucleotide” sequence disclosed herein include partial or full-length native TPP1 sequences, as with naturally occurring wild-type and functional polymorphic proteins, functional subsequences (fragments) thereof, and modified forms or sequence variants thereof, so long as the polypeptide retains some degree of TPP1 enzyme activity. Accordingly, in methods of the invention, such polypeptides encoded by nucleic acid sequences can be, but are not required to be, identical to the endogenous protein TPP1 protein that is defective, or whose expression is insufficient, or deficient in a treated mammal.

Amino acid changes in a polypeptide can be achieved by changing the codons of the corresponding nucleic acid sequence. Such polypeptides can be obtained based on substituting certain amino acids for other amino acids in the polypeptide structure in order to modify or improve biological activity. For example, through substitution of alternative amino acids, small conformational changes may be conferred upon a polypeptide that results in increased activity. Alternatively, amino acid substitutions in certain polypeptides may be used to provide residues, which may then be linked to other molecules to provide peptide-molecule conjugates which, retain sufficient properties of the starting polypeptide to be useful for other purposes.

One can use the hydropathic index of amino acids in conferring interactive biological function on a polypeptide, wherein it is found that certain amino acids may be substituted for other amino acids having similar hydropathic indices and still retain a similar biological activity. Alternatively, substitution of like amino acids may be made on the basis of hydrophilicity, particularly where the biological function desired in the polypeptide to be generated in intended for use in immunological embodiments. The greatest local average hydrophilicity of a “protein”, as governed by the hydrophilicity of its adjacent amino acids, correlates with its immunogenicity. Accordingly, it is noted that substitutions can be made based on the hydrophilicity assigned to each amino acid.

In using either the hydrophilicity index or hydropathic index, which assigns values to each amino acid, conduct substitutions of amino acids where these values are ±2, with ±1 being typical, and those with in ±0.5 being the most typical substitutions.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues). One non-limiting example of a nucleic acid modification is codon optimization.

An example of an amino acid modification is a conservative amino acid substitution or a deletion. In particular embodiments, a modified or variant sequence (e.g., TPP1) retains at least part of a function or activity of the unmodified sequence (e.g., wild-type TPP1).

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of enzymatic activity of the wild-type TPP1).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of enzymatic activity of the wild type TPP1).

“Conservatively modified variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are substantially identical is that one polypeptide is immunologically reactive with antibodies raised against the second polypeptide. Thus, a polypeptide is substantially identical to a second polypeptide, for example, where the two peptides differ only by a conservative substitution.

The term “about” at used herein refers to a values that is within 10% (plus or minus) of a reference value.

The terms “treat” and “treatment” refer to both therapeutic treatment and prophylactic or preventative measures, wherein the object is to prevent or decrease an undesired physiological change or disorder, such as the development or spread of cancer. For purposes of this invention, beneficial or desired clinical results include, but are not limited to, alleviation of symptoms, diminishment of extent of disease, stabilizing a (i.e., not worsening or progressing) symptom or state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The terms “a” and “an” and “the” and similar referents in the context of describing the invention are to be construed to cover both the singular and the plural, unless otherwise indicated herein or clearly contradicted by context. Thus, for example, reference to “a vector” includes a plurality of such vectors, reference to “a virus” or “particle” includes a plurality of such virions/particles and reference to “AAV or rAAV particle” includes a plurality of such AAV or rAAV particles.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All applications, publications, patents and other references, GenBank citations and ATCC citations cited herein are incorporated by reference in their entirety. In case of conflict, the specification, including definitions, will control.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features.

All numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

Embodiments of the invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the elements in all possible variations thereof is encompassed by the invention unless otherwise indicated herein or otherwise clearly contradicted by context. Accordingly, the following examples are intended to illustrate but not limit the scope of the invention claimed.

### EXAMPLE 1

Human TPP1 Sequences

| Human TPP1 Protein Sequence (SEQ ID NO: 1): | |
|---|---|
| MGLQACLLGLFALILSGKCSYSPEPDQRRTLPPGWVSLGRADPEEELSLTFALRQQNVERLSELVQAVSDPSSPQYG | |
| KYLTLENVADLVRPSPLTLHTVQKWLLAAGAQKCHSVITQDFLTCWLSIRQAELLLPGAEFHHYVGGPTETHVVRSP | |
| HPYQLPQALAPHVDFVGGLHHFPPTSSLRQRPEPQVTGTVGLHLGVTPSVIRKRYNLTSQDVGSGTSNNSQACAQFL | |
| EQYFHDSDLAQFMRLFGGNFAHQASVARVVGQQGRGRAGIEASLDVQYLMSAGANISTWVYSSPGRHEGQEPFLQWL | |
| MLLSNESALPHVHTVSYGDDEDSLSSAYIQRVNTELMKAAARGLTLLFASGDSGAGCWSVSGRHQFRPTFPASSPYV | |
| TTVGGTSFQEPFLITNEIVDYISGGGFSNVFPRPSYQEEAVTKFLSSSPHLPPSSYFNASGRAYPDVAALSDGYWVV | |
| SNRVPIPWVSGTSASTPVFGGILSLINEHRILSGRPPLGFLNPRLYQQHGAGLFDYTRGCHESCLDEEVEGQGFCSG | |
| PGWDPVTGWGTPNFPALLKTLLNP | |
| Human TPP1 Nucleic Acid Sequence (SEQ ID NO: 2): | |
| 1 | cgcggaaggg cagaatggga ctccaagcct gcctcctagg gctctttgcc ctcatcctct |
| 61 | ctggcaaatg cagttacagc ccggagcccg accagcggag gacgctgccc ccaggctggg |
| 121 | tgtccctggg ccgtgcggac cctgaggaag agctgagtct cacctttgcc ctgagacagc |
| 181 | agaatgtgga aagactctcg gagctggtgc aggctgtgtc ggatcccagc tctcctcaat |
| 241 | acggaaaata cctgacccta gagaatgtgg ctgatctggt gaggccatcc ccactgaccc |
| 301 | tccacacggt gcaaaaatgg ctcttggcag ccggagccca gaagtgccat tctgtgatca |
| 361 | cacaggactt tctgacttgc tggctgagca tccgacaagc agagctgctg ctccctgggg |
| 421 | ctgagtttca tcactatgtg ggaggaccta cggaaaccca tgttgtaagg tccccacatc |
| 481 | cctaccagct tccacaggcc ttggcccccc atgtggactt tgtgggggga ctgcaccatt |
| 541 | ttcccccaac atcatccctg aggcaacgtc ctgagccgca ggtgacaggq actgtaggcc |
| 601 | tgcatctggg ggtaaccccc tctgtgatcc gtaagcgata caacttgacc tcacaagacg |
| 661 | tgggctctag caccagcaat aacagccaag cctgtgccca gttcctggag cagtatttcc |
| 721 | atgactcaga cctggctcag ttcatgcgcc tcttcggtgg caactttgca catcaggcat |
| 781 | cagtagcccg tgtggttgga caacagggcc ggggccgggc cgggattgag gccagtctag |
| 841 | atgtgcagta cctgatgagt gctggtgcca acatctccac ctgggtctac agtagccctg |
| 901 | gccggcatga gggacaggag ccottcctgc agtggctcat gctgctcagt aatgagtcag |
| 961 | ccctgccaca tgtgcatact gtgagctatg gagatgatga ggactccctc agcagcgcct |
| 1021 | acatccagcg ggtcaacact gagctcatga aggctgctgc tcggggtctc accctgctct |
| 1081 | tcgcctcagg tgacagtggg gccgggtgtt ggtctgtctc tggaagacac cagttccgcc |
| 1141 | ctaccttccc tgcctccagc ccctatgtca ccacagtggg aggcacatcc ttccaggaac |
| 1201 | ctttcctcat cacaaatgaa attgttgact atatcagtgg tggtggcttc agcaatgtgt |
| 1261 | tcccacggcc ttcataccag gaggaagctg taacgaagtt cctgagctct agcccccacc |
| 1321 | tgccaccatc cagttacttc aatgccagtg gccgtgccta cccagatgtg gctgcacttt |
| 1381 | ctgatggcta ctgggtggtc agcaacagag tgcccattcc atgggtgtcc ggaacctcgg |
| 1441 | cctctactcc agtgtttggg gggatcctat ccttgatcaa tgagcacagg atccttagtg |
| 1501 | gccgcccccc tcttgqcttt ctcaacccaa ggctctacca gcagcatggg gcaggactct |
| 1561 | ttgatgtaac ccgtggctgc catgagtcct gtctggatga agaggtagag ggccagggtt |
| 1621 | tctgctctgg tcctggctgg gatcctgtaa caggctgggg aacacccaac ttcccagctt |
| 1681 | tgctgaagac tctactcaac ccctgaccct ttcctatcag gagagatggc ttgtcccctg |
| 1741 | ccctgaagct ggcagttcag tcccttattc tgccctgttg gaagccctgc tgaaccctca |
| 1801 | actattgact gctgcagaca gcttatctcc ctaaccctga aatgctgtga gcttgacttg |
| 1861 | actcccaacc ctaccatgct ccatcatact caggtctccc tactcctgcc ttagattcct |
| 1921 | caataagatg ctgtaactag cattttttga atgcctctcc ctccgcatct catctttctc |
| 1981 | ttttcaatca ggcttttcca aagggttgta tacagactct gtgcactatt tcacttgata |
| 2041 | ttcattcccc aattcactgc aaggagacct ctactgtcac cgtttactct ttcctaccct |
| 2101 | gacatccaga aacaatggcc tccagtgcat acttctcaat ctttgcttta tggcctttcc |
| 2161 | atcatagttg cccactccct ctccttactt agcttccagg tcttaacttc tctgactact |
| 2221 | cttgtcttcc tctctcatca atttctgctt cttcatggaa tgctgacctt cattgctcca |
| 2281 | tttgtagatt tttgctcttc tcagtttact cattgtcccc tggaacaaat cactgacatc |
| 2341 | tacaaccatt accatctcac taaataagac tttctatcca ataatgattg atacctcaaa |
| 2401 | tgtaagatgc gtgatactca acatttcatc gtccaccttc ccaaccccaa acaattccat |
| 2461 | ctcgtttatt cttggtaaat gatgctatgc tttttccaac caagccagaa acctgtgtca |
| 2521 | tcttttcacc ccaccttcaa tcaacaagtc ctcaatcaac aagtcctact gactgcacat |
| 2581 | cttaaatata tctttatcag tccacaagtc attccaatta tatttcccaa gtatatctag |
| 2641 | aacttatcca cttatatccc cactgctact accttagttt agggctatat tctcttgaaa |
| 2701 | aaaagtgtcc ttacttcctg ccaatcccca agtcatcttc cagagtaaaa tgcaaatccc |
| 2761 | atcaggccac ttggatgaaa acccttcaag gattactgga tagaattcag gatttcccct |
| 2821 | ccagccccca atcatagctc acaaaccttc cttgctattt gttcttaagt aaaaaatcat |
| 2881 | ttttcctcct ccctccccaa accccaagga actctcactc ttgctcaagc tgttccgtcc |
| 2941 | ccttaccacc cctgatacaa ctgccaggtt aatttccaga attcttgcaa gactcagttc |
| 3001 | agaagtcacc ttctttcgtg aatgttttga ttccctgagg ctactttatt ttggtatggc |
| 3061 | tgaaaaatcc tagattttct aaacaaaacc tgtttgaatc ttggttctga tatggactag |
| 3121 | gagagagact gggtcaagta agcttatctc cctgaggctg tttcctcgtc tgttaagtgt |
| 3181 | gaatatcaat acctgccttt cataatcacc agggaataaa gtggaataat gttgataaca |
| 3241 | gtgcttggca cctggaagta ggtggcagat gttaacgccc ttcctccctt gcactgcgcc |
| 3301 | ccctgtgcct acctctagca ttgtaacgac cacatagtat tgaaatggcc agtttacttg |
| 3361 | tctgccttcc tttccaagac cgttggtgcc tagaggacta gaatcgtgtc ctatttaact |
| 3421 | ttgtgttccc aggtcctagc tcaggagttg gcaaataaga attaaatgtc tgctacaccg |
| 3481 | aaacaaa |

AAV Vector Production: Recombinant AAV4 vectors expressing human TPP1 under control of the CMV early enhancer/chicken β-actin (CAG) promoter were generated using standard triple transfection methods and purification by CsCl gradient centrifugation (Wright, 2008; Wright, 2009). Titers were quantified by silver stain after gel electrophoresis (SDS-PAGE) and PCR (Wright, 2008; Wright, 2009)

### EXAMPLE 2

TPPI activity in Tissue samples. TPP1 activity was assayed using a modified method described previously (Sohar, I., et al. 2000. Clin Chem, 46:1005-8). Briefly, samples were homogenized with a laboratory homogenizer (P200; Pro Scientific, Oxford, Conn.) in 2000 ice-cold homogenization buffer-0.1% Triton X-100 in normal saline with Complete Protease Inhibitor Cocktail (Roche, Mannheim, Germany). Insoluble material was removed from the homogenate by centrifugation at 21×10³ rcf at 4° C.×15 minutes, and protein content in the supernatant was quantified by DC Protein Assay (Biorad, Hercules, Calif.). Protein (10 μl) was added to wells of a 96-well black wall plate containing 90 μl of 100 mM sodium citrate buffer, 150 mM NaCl, and 0.1% triton X-100 (pH 4.0) with the enzyme substrate (250 μmol/l Ala-Ala-Phe 7-amido-4-methylcoumarin in sodium citrate buffer, pH 4.0). Plates were quantified using a SpectraMax M5 microplate reader (Molecular Devices, Sunnyvale, Calif.) at 37° C. with 355±9 nm excitation wavelength and 460±15 nm emission wavelength. Purified recombinant human TPP1 (generous gift from P. Lobel, State University of New Jersey, N.J.) was used as a standard.

TPPI activity in Cerebrospinal fluid (CSF). TPP1 proenzyme was activated as described in (Tian, Y., et al. 2006. J Biol Chem, 281:6559-72). CSF from the animals in the same study group was pooled and incubated with the same volume of 100 mM acetate buffer, 150 mM NaCl and 0.1% triton X-100 (pH 4.0) overnight at room temperature. Activated TPP1 was quantified as in tissue samples.

Intraventricular AAV vector administration. Animals were fully anesthetized. The injection site was shaved and mice mounted into a Kopf stereotaxic rig and continued to receive 2% Isoflurane/oxygen mixture through a nose cone. An analgesic (meloxicam, 1-10 mg/kg) was administered sub-cutaneously pre-incision. Ophthalmic ointment was topically applied to prevent eyes drying during surgery. A 10 or 25 μl Hamilton syringe with 0.4″ 33GA Hamilton needle was primed by drawing up and expulsing the test article 20 times; the test article was discarded. The syringe was loaded with fresh test article and then loaded into the injector (Stoelting Company). The site of incision was cleaned. The injection site was calculated from Bregma (+0.3 mm anterior, −1.0 mm lateral) then a burr hole made in the cranium. The needle with test article was then lowered −2.0 mm deep from the surface of the brain, the test article infused at a rate of 200 nl/min and the needle was allowed to dwell for 5 minutes after infusion before retraction. The incision was closed using non-absorbable sutures. Analgesic (Bupivicaine HCl, 0.5%) was topically applied pre-emergence from anesthesia to reduce pain and discomfort after surgery.

Tremor Assay. Differences in tremor amplitude between normal and CLN2^{-/-} mice have been observed, which can be prevented by enzyme replacement therapy (Chang, M., et al. 2008. Mol Ther, 16:649-56; Chen, Y. H., et al. 2009. Nat Med, 15:1215-8). For the current study, tremor assay was performed in animals 5 weeks post-injection. Wild type and uninjected CLN2^{-/-} mice included in the study served as controls. The tremor assay was carried out with a Tremor Monitor System (San Diego Instruments, San Diego, Calif.). The Tremor Monitoring System is a cabinet, which provides sound attenuation and visual isolation, equipped with an enclosure cylinder that sits on a platform with a piezoelectric sensor. This sensor transforms animal tremors into an electrical current. The signal is amplified in the Tremor Monitor equipment bay and sent to a computer for recording. Recordings were made at 128 samples per second and 200-206 mV of gain. After 3 minutes of habituation, animal tremor was recorded during 5 minutes. Tremor signal was quantified as amplitude (dBV) respect to frequency (Hz). Statistical analysis consisted in a 2-way ANOVA followed by a Sidak's multiple comparison test as post-hoc test.

CSF Collection. Animals were placed into an Isoflurane induction chamber and exposed to 2.5% Isoflurane/oxygen mixture until fully anesthetized and exhibited no pedal response. The injection site was shaved and mice were mounted into a RWD Life Science (Shenzhen, China) stereotaxic rig and continued to receive 2% Isoflurane/oxygen mixture through a nose cone. The membrane over the Cisterna Magna was exposed by an incision along the neck and skin and neck muscles separated. A glass capillary was inserted into the cisterna magna and CSF was collected by capillarity for 20 minutes.

After CSF collection, animals were sedated with 5% isoflurane/oxygen mix prior to and during euthanized by transcardial perfusion with 20 ml of ice-cold PBS until the liver became a light coffee color. Tissues were harvested for post-necropsy analysis. All tissue samples were flash frozen immediately after collection, and stored at −80° C.

AAV vector Biodistribution. Genomic DNA was collected from flash frozen brain and peripheral tissues using a Qiagen DNA extractor kit (Qiagen, Valencia, Calif.). AAV4 copies/mg of DNA were determined by Q-PCR of vector-specific sequences for each sample, in triplicate. Forward primer probe sequence (5′-FAM/ATTGTCCAA/ZEN/GCTGGTACAGGCTGT/3IABkFQ) and reverse primer probe sequence (CTTTCTCAACCCAAGGCTCTA) were synthetized by Integrated DNA Technologies (Coralville, Iowa). 10 μl reaction (5.5 μl mastermix+4.5 μl sample) were run in a CFX384 Real-Time System (BioRad, Hercules, Calif.) at 95° C. for 10 minutes followed by 39 cycles of 95° C. for 15 seconds and 60° C. for 1 minute. The AAV4 genome copies from the samples were calculated using a standard curve made from the AAV4 human TPP1 plasmid DNA (10e3 to 10e10 copies/ml). The final AAV4 genome copies (vg)/mg of DNA in the samples was calculated by the formula:

AAV4 genome copies (vg)/mg DNA=2*(AAV4 copies/ml)ₛₐₘₚₗₑ/(mg DNA/ml)ₛₐₘₚₗₑ

Correction factor of 2 in the previous formula is for correcting the conversion from dsDNA plasmid, used for the standard curve, to a ssDNA in the AAV4 vectors.

### EXAMPLE 3

Dose-response and stability studies were performed using the CLN2 knock out (CLN^{-/-}) mouse model, which does not express TPP1. The dose-response study included 30 mice (16 female, 14 male) between 5 and 8 weeks of age at the time of injection. The stability study included 14 mice (7 female, 7 male) between 6 and 8 weeks of age at the time of injection.

TABLE 1
Dose-Response Study Design

| Dose | N | Weeks post- |
|---|---|---|
| (vg) | (female; male) | injection |
| 1e10 | 12 (6 ♀; 6♂) | 5 |
| 5e10 | 9 (6 ♀; 3♂) | 5 |
| 1e11 | 10 (5 ♀; 5♂) | 5 |

TABLE 2
Stability Study Design

| Dose | N | Weeks post- |
|---|---|---|
| (vg) | (female; male) | injection |
| 5e10 | 4 (2 ♀; 2♂) | 3 |
| 5e10 | 5 (3 ♀; 2♂) | 9 |
| 5e10 | 5 (1 ♀; 4♂) | 12 |

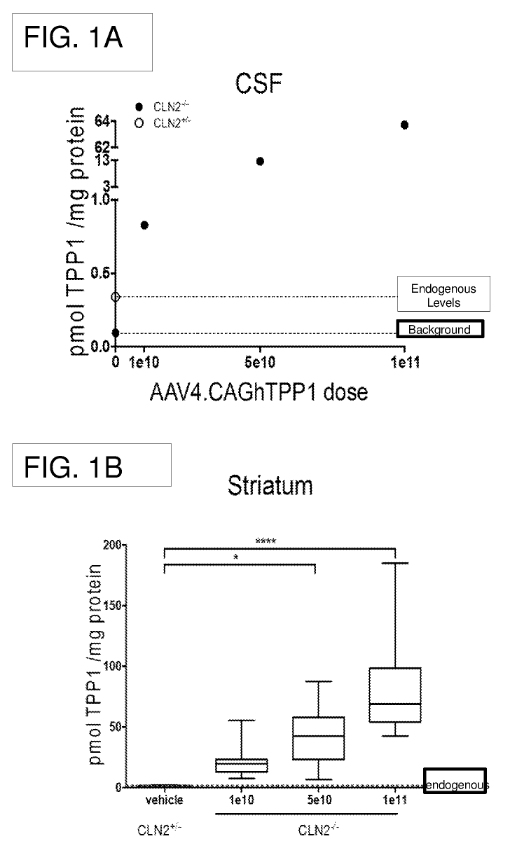

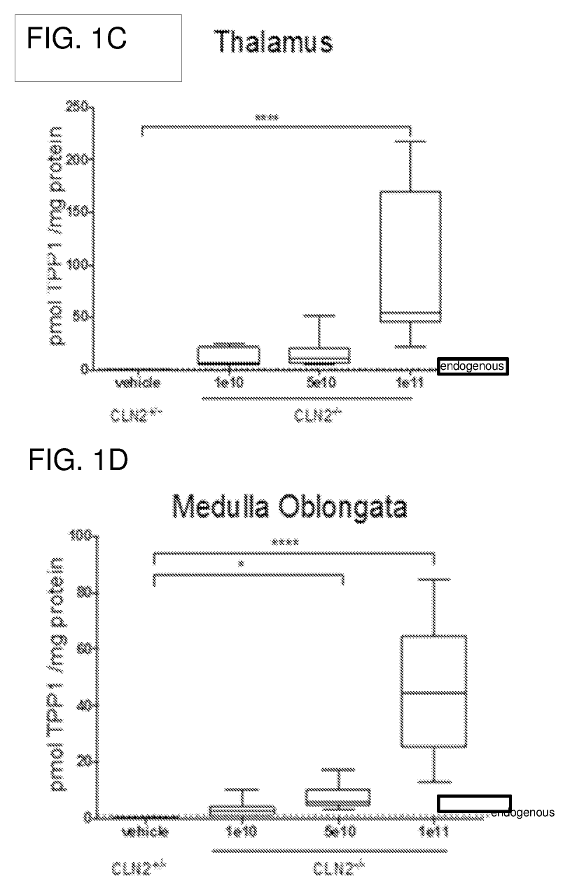

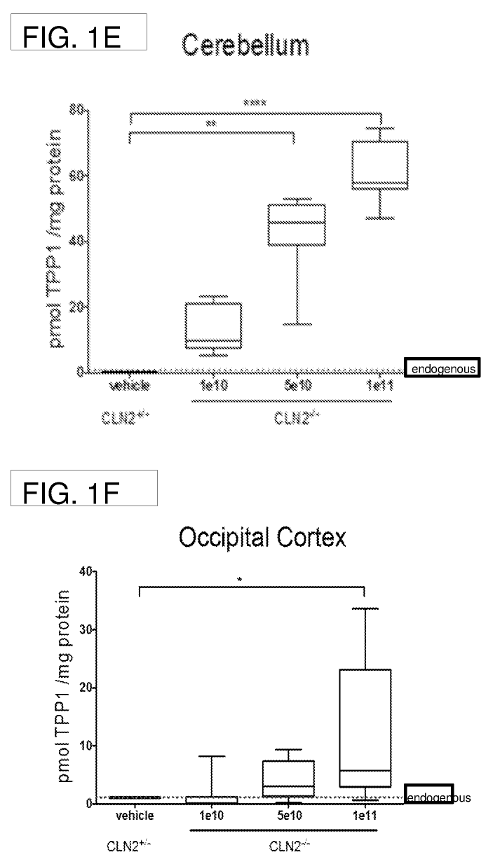

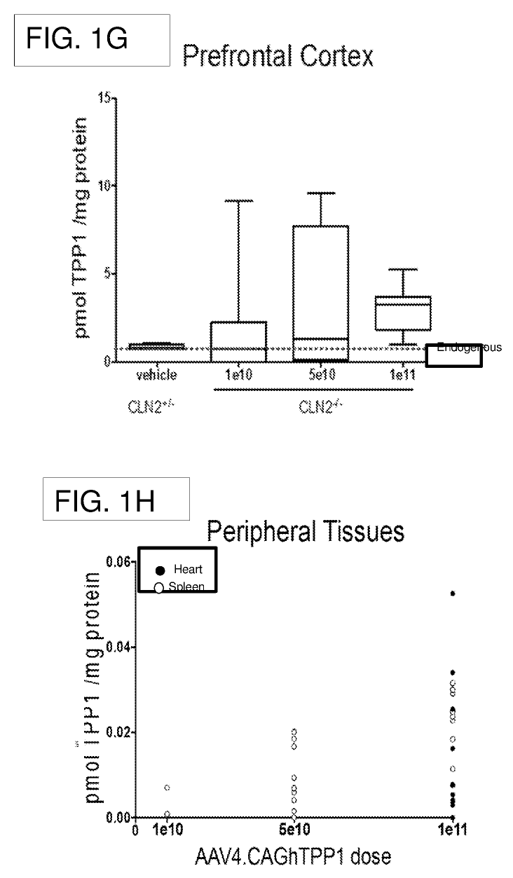

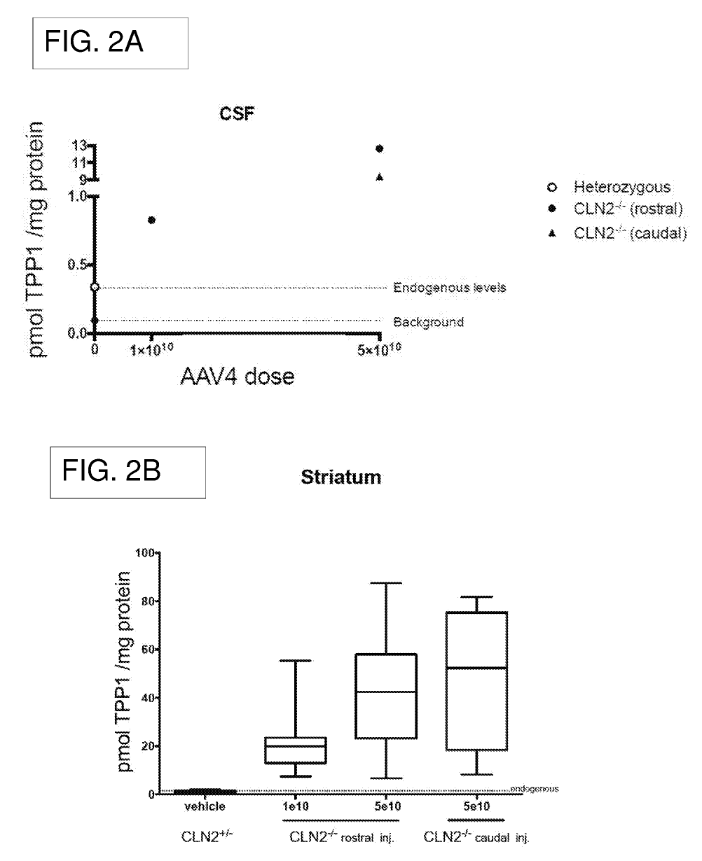

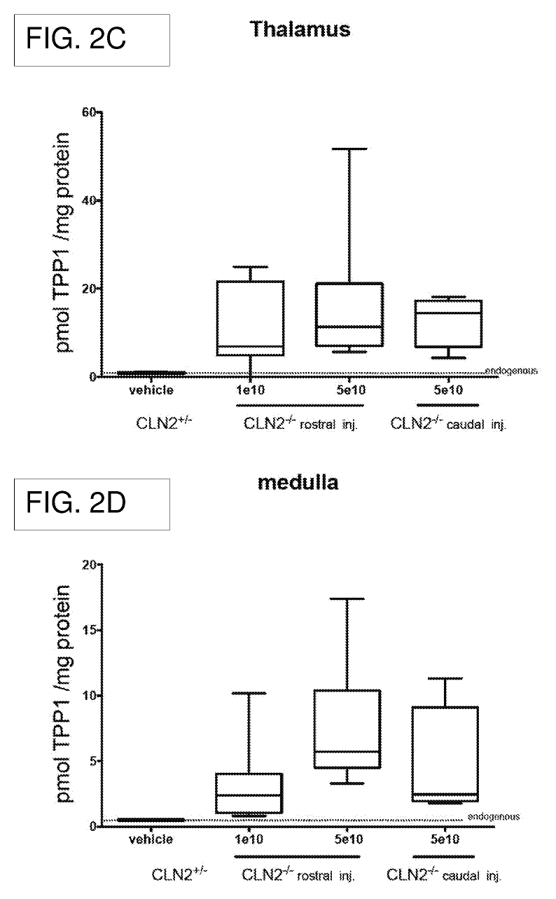

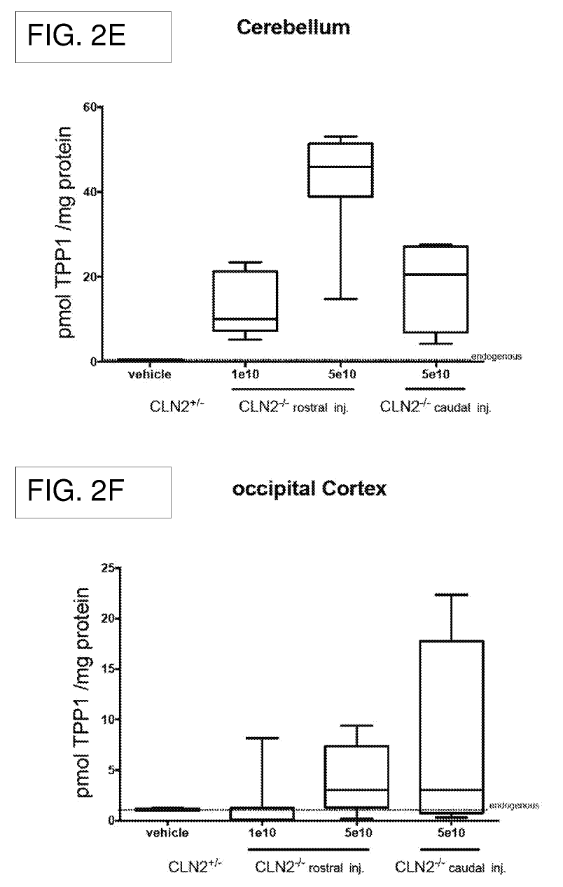

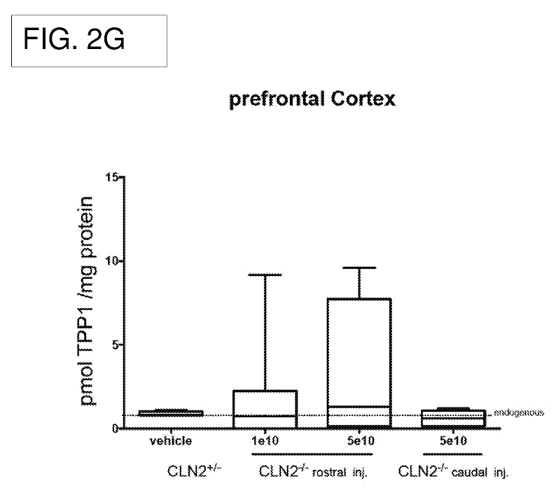

Mice were injected with AAV4.CAGhTPP1 at doses of 1e10, 5e10, or 1e11 into the rostral aspect of the right lateral ventricle as described above. Injections were performed using a stereotaxic mouse frame, and injection coordinates from the bregma point were fixed as +0.3mm anterior, −1 mm lateral, −2 mm deep from the pia using The sterotaxic mouse brain atlas by G. Paxinos and K. B. J. Franklin (2^{nd} edition, Academic Press, 2001). This injection point was called “rostral injection” (FIGS. 1A-1H and 2A-2G).

For the dosing study, test article was injected at escalating doses of 1e10 vg in 10 μl, 5e10 vg in 10 μl (RVC302), and 1e11 vg in 15 μl; mice were kept 5 weeks post-injection. For the stability study, all animals were injected with 5e10 vg and euthanized 3, 9, or 12 weeks post-injection.

The test article was thawed and diluted with excipient immediately prior to intraventricular injections. The excipient was PBS180-F69 pH 7.4 (0.01M Na₂HPO₄, 0.18M NaCl, 0.001% Pluronic F-68).

In the dosing study, mice were assayed for tremor behavior prior to euthanasia. For both studies cerebral spinal fluid (CSF) was collected and pooled within groups before intracardiac perfusion with ice-cold PBS. Brain tissue (striatum, thalamus, medulla, cerebellum, occipital cortex, and pre-frontal cortex), heart, spleen, liver and kidney were collected for molecular analysis. Tissue samples and CSF were used for enzyme activity quantification and/or biodistribution assays.

Five weeks post-injection for both studies, animals were anesthetized and cerebrospinal fluid (CSF) was collected from the cisterna magna.

After euthanasia, blood was cleared by ice-cold PBS intracardiac perfusion, and different brain areas (CSF, striatum, thalamus, medulla oblongata, cerebellum, occipital and prefrontal cortex, FIGS. 1A-1H and 2A-2G) from both hemispheres were collected for TPP1 quantification by TPP1 activity assay, and AAV4 biodistribution analyses by QPCR.

To analyze ependyma transduction improvement by altering coordinates, 5e10 vg of AAV4.CAGhTPP1 was infused into the lateral ventricle at a more caudal point (from bregma; −2.18 mm anterior, −2.9 mm lateral, −3.5 mm deep from bone). Samples from these animals are noted as “caudal injection.” Samples were collected as described above for TPP1 quantification by TPP1 activity assay.

Expression of TPP1 in the different brain areas for caudal and rostral injection (CSF, striatum, thalamus, medulla oblongata, cerebellum, occipital and prefrontal cortex) are shown in FIGS. 2A-2G.

### EXAMPLE 4

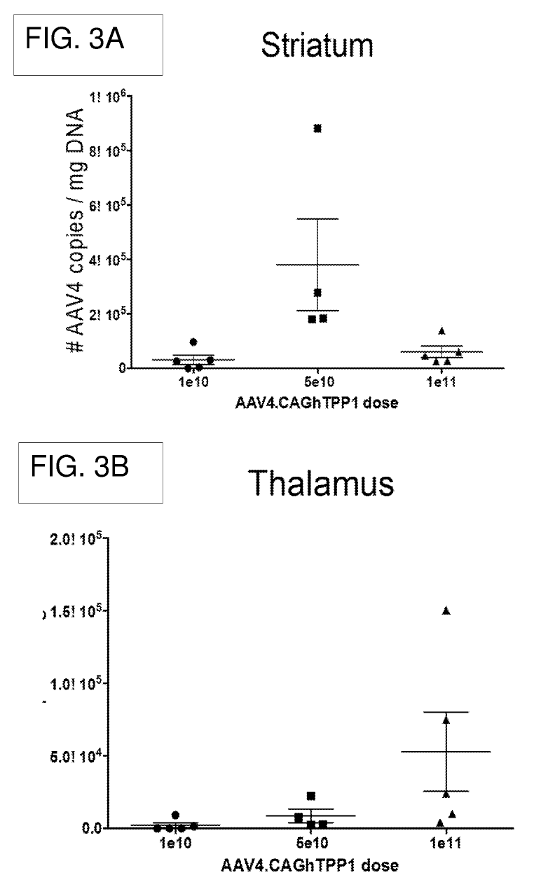

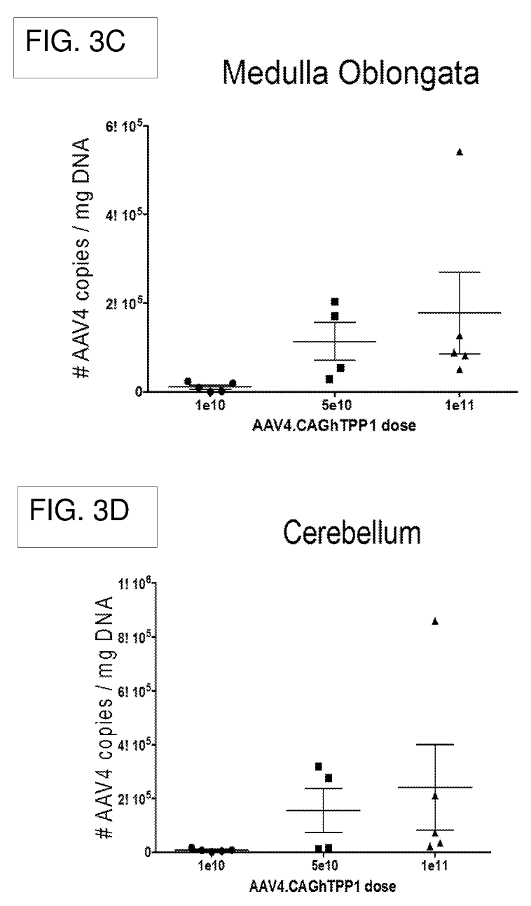

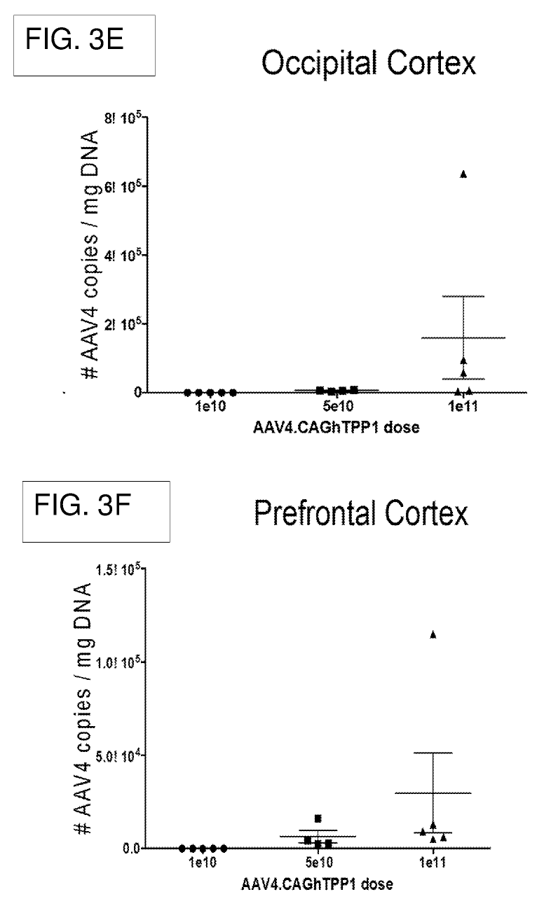

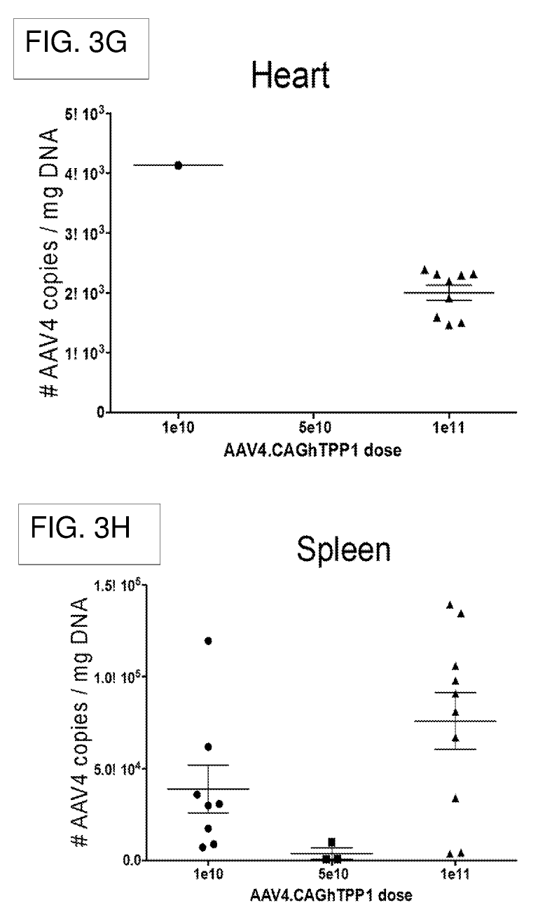

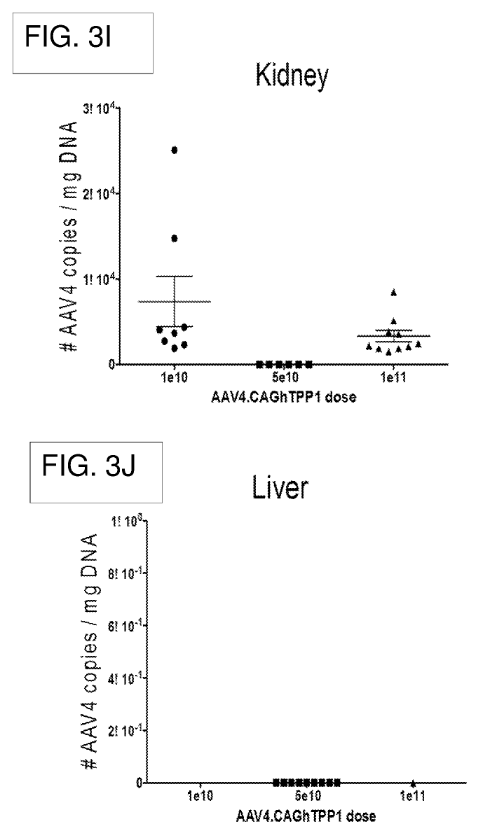

Dosing Study Summary. Human TPP1 (hTPP1) enzyme activity assays were conducted on the CSF, brain, and peripheral samples. CSF was pooled from all animals in each treatment group. The results show a clear dose response relative to endogenous murine TPP1 levels (0.34 pmol/mg protein; FIG. 3). Recombinant TPP1 levels were 0.83, 12.66, and 63.70 pmol TPP1/mg of protein for 1e10, 5e10, and 1e11 vg doses respectively.

In most brain areas analyzed, recombinant TPP1 levels were higher than endogenous murine TPP1 and parenchymal concentrations increased in a dose-dependent manner. However, the prefrontal cortex did not display a dose-dependent increase. The prefrontal cortex represents the brain region furthest from the site of injection and lacks ependymal cells. With the exception of the prefrontal cortex, all of the high dose animals had recombinant TPP1 levels significantly over endogenous TPP1 levels.

Post-mortem biodistribution analysis revealed AAV4 viral genomes in parenchymal punches that contained ependymal cells. Four of the high dose animals had viral genomes in cortical areas. Viral genomes were also found in the ependymal cells in the fourth ventricle. The broad distribution of viral vector throughout the brain could be due to the high ratio of injection volume to ventricular volume. For context, the mouse ventricular system has a volume of 15 μl (FIG. 1) and the injected volume was 10 μl for the low and middle dose, and 15 μl in the high dose groups.

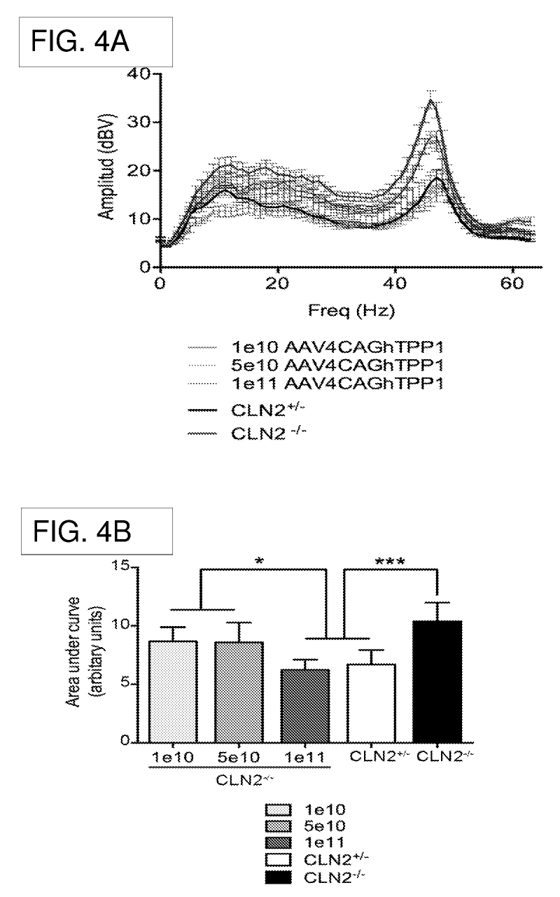

Post-mortem viral biodistribution analysis in the peripheral tissues displayed AAV4 genomes predominantly in the spleen of the low and high dose groups (FIG. 4). Viral genomes were found at lower levels in the kidney of both groups and in the hearts of the high dose group. No AAV4 viral genomes were found in the peripheral tissues of the middle dose group.

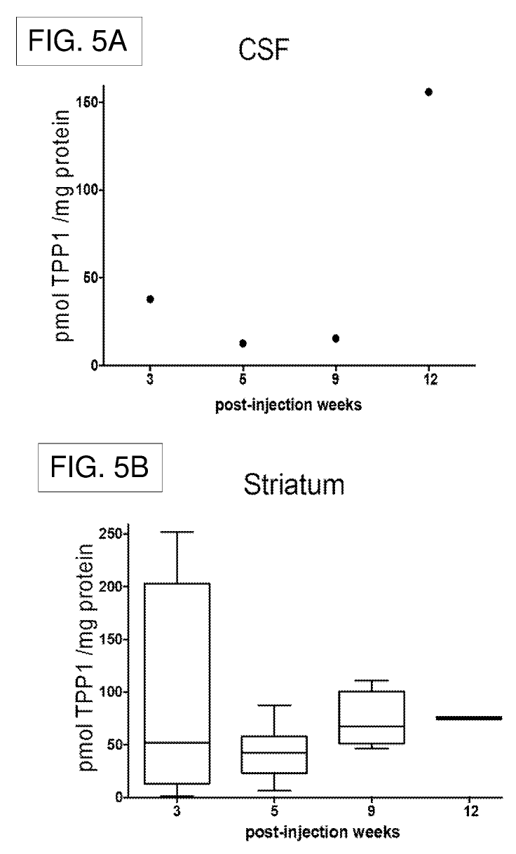

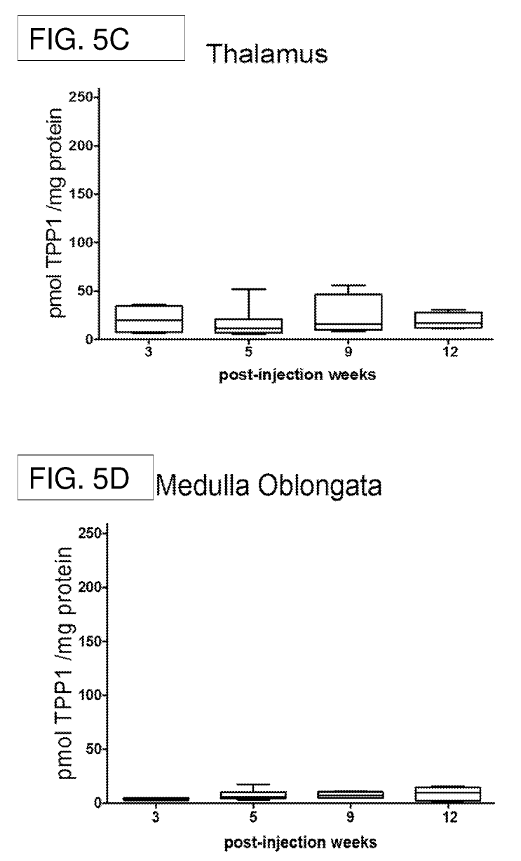

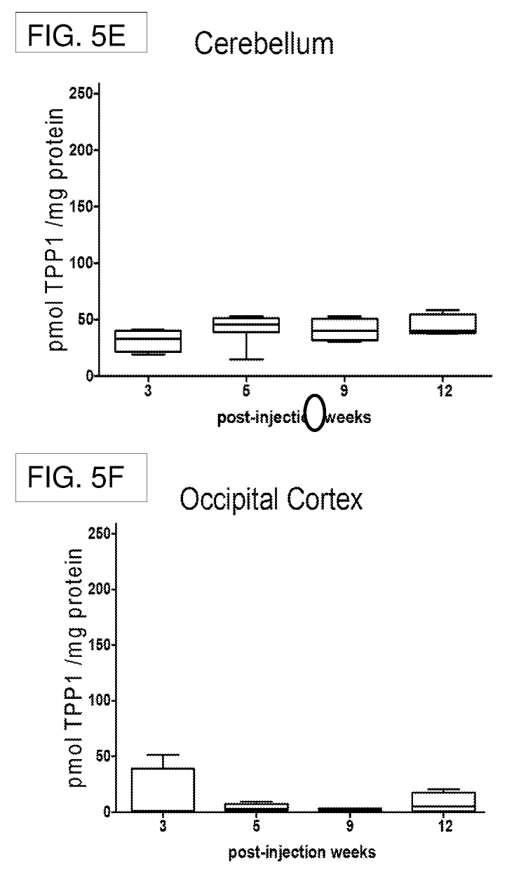

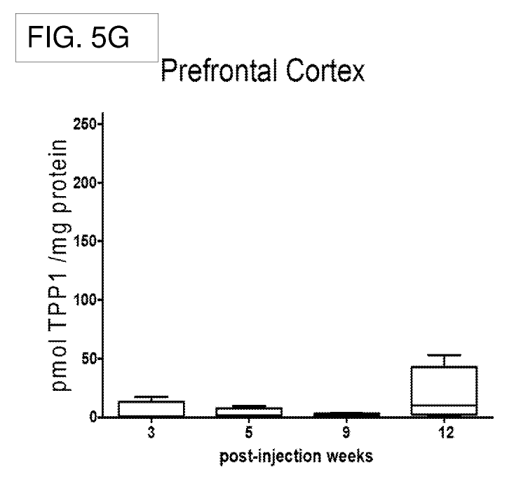

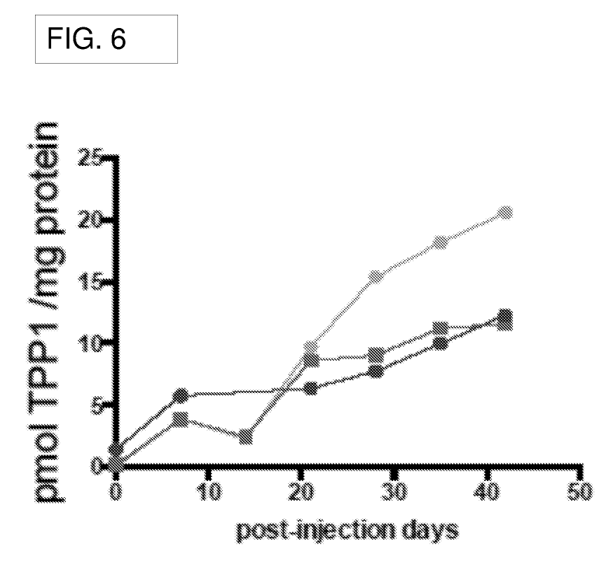

CLN2^{-/-} mice present with elevated tremor activity starting at 12 weeks and progresses with the disease (Chang, M., J. D. Cooper, D. E. Sleat, S. H. Cheng, J. C. Dodge, M. A. Passini, P. Lobel, and B. L. Davidson. 2008. Mol Ther, 16:649-56). Five weeks after AAV4CAGhTPP1 treatment (10-13 weeks of age) this characteristic tremor activity was decreased, with total prevention in the high dose group (FIG. 5).

Stability Study Summary. Stable expression of recombinant TPP1 was achieved with 5e10 vg AAV4CAGhTPP1 in all brain regions sampled until study termination (12 weeks post-injection). It is important to note that the average lifespan of untreated CLN2^{-/-} mice is 16 weeks. All of the mice in the 12 week post-injection group survived longer than untreated CLN2^{-/-} mice, until they were euthanized at 19 weeks of age.

### EXAMPLE 5

### CONCLUSIONS

Dosing Study. Recombinant TPP1 levels in CSF displayed a dose-response pattern five weeks after AAV4CAGhTPP1 injection.

Mature recombinant TPP1 was detected throughout the brain. Supraendogenous levels of hTPP1 were quantified in brain areas along the ventricles in a dose-response manner among the three experimental groups. All doses achieved endogenous levels in brain areas spatially distal to the site of infusion.

AAV4CAGhTPP1 vector was predominantly localized to parenchymal tissue punches containing ependymal cells. However, a small number of viral particles were found to transduce heart and spleen.

Behavioral measurements revealed a total prevention of the tremor phenotype in the high dose injected animals and a partial rescue of the tremor phenotype in the low and middle dose groups.

Stability Study. Supraendogenous levels of the hTPP1 proenzyme were maintained up to 12 weeks post-injection in CLN2^{-/-} mice receiving 5e10 vg. An unexpected but exciting result in these mice was an increase in life span.

To summarize, AAV4CAGhTPP1 was widely expressed in the brains of CLN2^{-/-} mice in a dose dependent manner. AAV4CAGhTPP1 was well tolerated and maintained stable expression levels 12 weeks post-injection and, furthermore, increased the normal lifespan of CLN2^{-/-} mice.

### EXAMPLE 6

### Post-Mortem Analysis

Dosing Study. All treatment groups expressed hTPP1 proenzyme levels in the CSF at higher than endogenous levels in CLN2^{+/-} animals (FIG. 1). There was a significant linear correlation (r²=0.91) for the three treatment groups, indicating a strong dose-response. CLN2^{-/-} mice injected with the high dose of AAV4CAGhTPP1 (1e11 vg) had ˜187-fold increase of proenzyme expression. The mid dose (5e10 vg) had ˜37-fold increase relative to endogenous levels. Most importantly, the low dose (1e10 vg) also achieved TPP1 proenzyme levels higher than endogenous levels (˜2.4-fold increase).

Parenchymal tissue punches from different regions throughout the brain were assayed for hTPP1 expression. In general, punches from tissue directly adjacent to the ventricular system (striatum, thalamus, cerebellum, medulla oblongata) expressed higher levels of hTPP1 relative to punches from the occipital and pre-frontal cortices. Significantly, all brain regions assayed from all treatment groups had TPP1 expression at least equivalent to the endogenous levels in CLN2^{+/-} mice. This indicates that a low dose of 1e10 vg of AAV4.CAGhTPP1 is sufficient to provide adequate levels of TPP1 in brain parenchyma (FIG. 1). Linear regression for each of these areas produced a statistically positive slope supporting a dose-response. Cerebellum and medulla oblongata were the most notable areas with r²=0.82, and 0.58 respectively. These data show accumulation of hTPP1 proenzyme in the IV ventricle, with better penetration in the cerebellar parenchyma than in medulla oblongata. In the high dose (1e 11 vg), there was a statistically significant increase in hTPP1 levels relative to endogenous levels in the striatum, thalamus, cerebellum, medulla oblongata, and occipital cortex. The mid dose (5e10 vg) also had statistically significant increases in hTPP1 levels in the striatum, cerebellum, and medulla oblongata.

Peripheral tissues including the heart, liver, kidney and spleen were analyzed for the presence of recombinant TPP1. All treatment groups had low, but detectable levels of TPP1 in the spleen (FIG. 1H). The analysis resulted in a statistical positive linear regression among the experimental groups (r²=0.54) reflecting a positive dose-response. Nine mice of the high dose group had recombinant enzyme in the heart at low concentration (less than 0.06 pmol TPP1/mg protein).

Stability Study. Stable expression of recombinant TPP1 was achieved with 5e10 vg AAV4.CAGhTPP1 in all brain regions sampled until study termination (12 weeks post-injection; FIG. 5). It is important to note that the average lifespan of untreated CLN2^{-/-} mice is 16 weeks. However, all of the mice in the stability study receiving 5e10 vg survived until euthanasia at 19 weeks of age. This data shows that AAV4.CAGhTPP1 treatment apparently also increases the life span of LINCL mice.

### Analysis of Biodistribution

Dosing Study. Q-PCR was performed using an AAV4CAGhTPP1-specific probe to analyze viral biodistribution. High concentrations of AAV4 viral genomes were localized to parenchymal punches that contained ependymal cells. Although variability was found among animals, there was a trend to express high levels of viral particles in the cerebellum and medulla oblongata. Both areas are associated with the IV ventricle, which is located distal from the injection point. These results indicate that the viral genomes (vg) are transported from the injection site to the IV ventricle by fast CSF flow (FIG. 3).

There was no statistical dose-response found in the brain when comparing among experimental groups. Surprisingly, no differences were detected between middle and high dose groups. In contrast, a lower number of viral genomes was quantified in peripheral tissues of the middle dose group, suggesting that most of the viral particles were retained in the brain at this dose. The spleen had the highest number of viral genomes of the peripheral tissues. Interestingly, all heart samples from the high dose had similar detectable levels of AAV4.CAGhTPP1.

### Tremor Assay

Dosing Study. Untreated 12 week old CLN2−/− mice exhibited enhanced tremor activity. Age matched CLN2^{-/-} mice that received 1e10 or 5e10 AAV4CAGhTPP1 had a significant decrease in the tremor amplitude at frequencies between 14-48 Hz (FIG. 4). The high (5e10) dose of AAV4CAGhTPP1 completely prevented the disease phenotype, as there was no difference in tremor amplitude of the high dose group compared with control CLN2^{+/-} mice.

### EXAMPLE 7

A study was performed in the Rhesus monkey to evaluate the expression profile of TPP1 in CSF over time following a single-dose intraventricular administration of AAV vectors expressing hTPP1. For that, 4 mL containing 3e13 vg of AAV2.CAGhTPP1 were unilaterally infused into the right occipital horn of the lateral ventricle of three Rhesus monkeys. CSF samples were collected before injection (day 0) and every 7 days. Animals were sacrificed 6 weeks after injection.

TPP1 levels in the cerebro-spinal fluid (CSF) were quantified by enzymatic assay and were normalized by mg of total protein. TPP1 concentration increased gradually to reach 9-20 fold over baseline levels by the end of the trial in all of the animals. These results show that a single injection of AAV2.CAGhTPP1 generates a robust increase of TPP1 level in the Rhesus CSF.

What is claimed is:

1. A method of treating a mammal having a lysosomal storage disease (LSD), said method comprising the steps:
administering to the brain or spine of a mammal a plurality of AAV particles,
said AAV particles comprising an
(i) AAV capsid protein;
(ii) a nucleic acid inserted between a pair of AAV inverted terminal repeats (ITRs), said nucleic acid encoding a polypeptide having lysosomal hydrolase activity;
(iii)an expression control element driving expression of said nucleic acid; and
said AAV particles being capable of transducing cells of said mammal and providing expression of said polypeptide.

2. A method according to claim 1, wherein said polypeptide has tripeptidyl-peptidase 1 (TPP1) activity.

3. A method according to claim 1, wherein said polypeptide comprises TPP1, a pro-enzyme thereof, or an enzymatically active variant thereof.

4. A method according to claim 1, wherein one or more of the AAV ITRs comprise one or more AAV2 ITRs.

5. A method according to claim 1, wherein the nucleic acid encodes mammalian TPP1.

6. A method according to claim 1, wherein the nucleic acid encodes human TPP1.

7. A method according to claim 1, wherein the nucleic acid encodes a protein with TPP1 activity and having 80% or more identity to human TPP1 set forth as SEQ ID NO:1.

8. A method according to any of claims 1-7, wherein the expression control element comprises a CMV enhancer.

9. A method according to any of claims 1-7, wherein the expression control element comprises a beta actin promoter.

10. A method according to any of claims 1-7, wherein the expression control element comprises a chicken beta actin promoter.

11. A method according to any of claims 1-7, wherein the expression control element comprises a CMV enhancer and a chicken beta actin promoter.

12. A method according to any of claims 1-7, wherein the expression control element comprises a sequence having 80% or more identity to CMV enhancer set forth in SEQ ID NO:3 and/or a sequence having 80% or more identity to chicken beta actin promoter set forth in SEQ ID NO:3.

13. A method according to any of claims 1-7, wherein the expression control element comprises a sequence having 80% or more identity to SEQ ID NO:3.

14. A method according to any of claims 1-7, wherein the expression control element comprises SEQ ID NO:3.

15. A method according to any of claims 1-14, wherein the capsid sequence comprises a VP1, VP2 and/or VP3 capsid sequence having 70% or more identity to AAV1, AAV2, AAV3, AAV4, AAV5, AAV6, AAV7, AAV8, AAV9, AAV10, AAV11, Rh10, Rh74 or AAV-2i8 VP1, VP2 and/or VP3 sequences.

16. A method according to any of claims 1-14, wherein the capsid sequence comprises a VP1 capsid sequence having 80% or more identity to AAV2, wherein the capsid sequence has a tyrosine at positions 444, 500 and/or 730 substituted with an amino acid that is not tyrosine.

17. A method according to any of claims 1-14, wherein the capsid sequence comprises a VP1 capsid sequence having 90% or more identity to AAV2, wherein the capsid sequence has a tyrosine at positions 444, 500 and/or 730 substituted with phenylalanine.

18. A method according to any of claims 1-14, wherein the capsid sequence comprises an AAV2 VP1 capsid sequence having a tyrosine at positions 444, 500 and/or 730 substituted with phenylalanine.

19. A method according to any of claims 1-14, wherein the capsid sequence comprises a VP1, VP2 or VP3 capsid sequence selected from any of: AAV1, AAV2, AAV3, AAV4, AAV5, AAV6, AAV7, AAV8, AAV9, AAV10, AAV11, Rh10, Rh74 or AAV-2i8 AAV serotypes.

20. A method according to any of claims 1-19, comprising administering the plurality of AAV particles to the brain of said mammal.

21. A method according to any of claims 1-19, comprising administering the plurality of AAV particles to the cisternae magna, intraventricular space, brain ventricle, subarachnoid space, intrathecal space and/or ependyma of said mammal.

22. A method according to any of claims 1-19, comprising administering the plurality of AAV particles to the cerebral spinal fluid (CSF) of said mammal.

23. A method according to any of claims 1-19, comprising administering the plurality of AAV particles to the ventricular system.

24. A method according to any of claims 1-19, comprising administering the plurality of AAV particles to the rostral lateral ventricle.

25. A method according to any of claims 1-19, comprising administering the plurality of AAV particles to the caudal lateral ventricle.

26. A method according to any of claims 1-19, comprising administering the plurality of AAV particles to the right and/or left lateral ventricle.

27. A method according to any of claims 1-19, comprising administering the plurality of AAV particles to the right and/or left rostral lateral ventricle.

28. A method according to any of claims 1-19, comprising administering the plurality of AAV particles to the right and/or left caudal lateral ventricle.

29. A method according to any of claims 1-28, wherein said AAV particles contact ependymal cells of said mammal.

30. A method according to any of claims 1-29, further comprising administering to said mammal a first immunosuppressive agent.

31. A method according to claim 30, further comprising administering to said mammal a second immunosuppressive agent.

32. A method according to claim 30 or 31, wherein at least one of said first immunosuppressive agent and said second immunosuppressive agent are administered to said mammal prior to administration of said AAV particles.

33. A method according to claim 30 or 31, wherein said first immunosuppressive agent is administered prior to administration of said AAV particles and said second immunosuppressive agent is administered prior to, concurrently with, or after administration of said AAV particles.

34. A method according to any of claims 30-33, wherein said first immunosuppressive agent comprises cyclosporine.

35. A method according claim 34, wherein said cyclosporine is administered at a dosage of about 5-20 mg/kg twice a day for a period of at least 3 months.

36. A method according to claim 34, wherein the dose of said cyclosporine administered is reduced after a 1-2 months after administration of said AAV particles.

37. A method according to any of claims 30-36, wherein said second immunosuppressive agent comprises mycophenolate or a derivative thereof.

38. A method according to claim 37, wherein said mycophenolate derivative is mycophenolate mofitil (MMF).

39. A method according to any of claims 30-38, wherein (i) said first immunosuppressive agent is administered at least about two weeks prior to administration of said AAV particles and (ii) said second immunosuppressive agent is administered about two weeks before or within 60 days after administration of said AAV particles.

40. A method according to any of claims 37-39, wherein said mycophenolate or a derivative thereof is administered at a dosage of about 5-20 mg/kg a day.

41. A method according to any of claims 1-40, wherein said AAV particles are administered at a dose of about 1×10⁸ to about 1×10¹⁵vg/kg.

42. A method according to any of claims 1-41, wherein cells comprising the cerebrospinal fluid (CSF) of said mammal are transduced by said AAV particles.

43. A method according to any of claims 1-42, wherein said AAV particles transduce ependymal cells of said mammal.

44. A method according to any of claims 1-43, wherein cells transduced with said AAV particles express and secrete said polypeptide into the CSF of said mammal.

45. A method according to any of claims 2-44, wherein, tripeptidyl-peptidase 1 (TPP1) activity in the cerebrospinal fluid of said mammal is detectable at a level of at least 5 pmol TPP1/mg protein, optionally for greater than 350 days.

46. A method according to any of claims 1-45, wherein said mammal is a non-rodent mammal.

47. A method according claim 46, wherein said non-rodent mammal is a primate.

48. A method according to claim 46, wherein said non-rodent mammal is a human.

49. A method according to claim 48, wherein said human is a child.

50. A method according to claim 49, wherein said child is from about 1 to about 4 years of age.

51. A method according to any of claims 1-50, wherein said LSD is infantile or late infantile ceroid lipofuscinoses (LINCL), neuronopathic Gaucher, Juvenile Batten, Fabry, MLD, Sanfilippo A, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type II/III, or Sandhoff disease.

52. A method according to any of claims 1-51, wherein administration of said AAV particles comprises injection of said AAV particles.

53. A method according to any of claims 1-52, wherein onset of a symptom associated with said LSD is delayed by 5-10, 10-25, 25-50 or 50-100 days.

54. A method according to claim 53, wherein said symptom is selected from the group consisting of proionceptive response, nystagmus, menace, pupillary light reflex, cerebellar ataxia and intention tremor.

55. A method according to any of claims 1-54, wherein measurable loss of cognitive function associated with said LSD is delayed by 5-10, 10-25, 25-50 or 50-100 days.

56. A method according to any of claims 1-55, wherein lifespan of a mammal having said LSD is extended by 5-10, 10-25, 25-50 or 50-100 days.

57. A method according to any of claims 1-56, wherein neutralizing antibodies are not detected in CSF of said mammal for at least 30, 60, 90, 120 or more days after administration of said AAV particles.

58. A method according to any of claims 1-57, wherein neutralizing antibodies are not detected in CSF of said mammal for at least 250 days after said administration of said AAV particles.

59. A method according to any of claims 1-58, wherein said polypeptide is expressed in the spleen or heart of said mammal.

60. A method according to any of claims 1-59, wherein said polypeptide is expressed in the striatum, thalamus, medulla, cerebellum, cerebrum, occipital cortex or prefrontal cortex of said mammal.

61. A method according to any of claims 1-60, wherein said expression control element provides greater expression of said nucleic acid or polypeptide than the CMV promoter in one or more of the striatum, thalamus, medulla, cerebellum, cerebrum, occipital cortex or prefrontal cortex of said mammal.

62. A method according to any of claims 1-61, wherein said expression control element provides about 1-4-fold greater expression of said nucleic acid or polypeptide than the CMV promoter in one or more of the striatum, thalamus, medulla, cerebellum, cerebrum, occipital cortex or prefrontal cortex of said mammal.

63. A method according to any of claims 1-62, wherein said expression control element provides about 1-2-fold greater expression of said nucleic acid or polypeptide than the CMV promoter in one or more of the striatum, thalamus, medulla, cerebellum, cerebrum, occipital cortex or prefrontal cortex of said mammal.

64. A method according to any of claims 1-63, wherein said AAV particles are selected from the group consisting of AAV1, AAV2, AAV3, AAV4, AAV5, AAV6, AAV7, AAV8, AAV9, AAV10, AAV11, AAV12, AAV-rh74, AAV-rh10 and AAV-2i8 particles.

65. A method according to any of claims 1-64, wherein one or more of said ITRs is selected from the group consisting of an AAV1, AAV2, AAV3, AAV4, AAV5, AAV6, AAV7, AAV8, AAV9, AAV10, AAV11, AAV12, AAV-rh74, AAV-rh10 and AAV-2i8 ITR.

66. A method according to any of claims 1-65, wherein the capsid sequence comprises a VP1, VP2 and/or VP3 capsid sequence having 90% or more identity to AAV1, AAV2, AAV3, AAV4, AAV5, AAV6, AAV7, AAV8, AAV9, AAV10, AAV11, Rh10, Rh74 or AAV-2i8 VP1, VP2 and/or VP3 sequences.

67. A method according to any of claims 1-66, wherein the capsid sequence comprises a VP1, VP2 or VP3 capsid sequence selected from any of: AAV1, AAV2, AAV3, AAV4, AAV5, AAV6, AAV7, AAV8, AAV9, AAV10, AAV11, Rh10, Rh74 or AAV-2i8 AAV serotypes.


SEQUENCE LISTING (5 sequences)

SEQ ID NO: 1 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHHFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDYTRGCHESCLDEEVEGQGFCSGPGWDPVTGWGTPNFPALLKTLLNP

SEQ ID NO: 2 - DNA, 3487 bp, Homo sapiens
cgcggaagggcagaatgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccattttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgctgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgctgtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaagccagaaacctgtgtcatcttttcaccccaccttcaatcaacaagtcctcaatcaacaagtcctactgactgcacatcttaaatatatctttatcagtccacaagtccttccaattatatttcccaagtatatctagaacttatccacttatatccccactgctactaccttagtttagggctatattctcttgaaaaaaagtgtccttacttcctgccaatccccaagtcatcttccagagtaaaatgcaaatcccatcaggccacttggatgaaaacccttcaaggattactggatagaattcaggctttcccctccagcccccaatcatagctcacaaaccttccttgctatttgttcttaagtaaaaaatcatttttcctcctccctccccaaaccccaaggaactctcactcttgctcaagctgttccgtccccttaccacccctgatacaactgccaggttaatttccagaattcttgcaagactcagttcagaagtcaccttctttcgtgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcactgcgccccctgtgcctacctctagcattgtaacgaccacatagtattgaaatggccagtttacttgtctgccttcctttccaagaccgttggtgcctagaggactagaatcgtgtcctatttaactttgtgttcccaggtcctagctcaggagttggcaaataagaattaaatgtctgctacaccgaaacaaa

SEQ ID NO: 3 - DNA, 1672 bp, Artificial Sequence
  CAG Promoter
atagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 4 - DNA, 15 bp, Artificial Sequence
  Forward Primer Probe
gctggtacaggctgt

SEQ ID NO: 5 - DNA, 21 bp, Artificial Sequence
  Reverse Primer Probe
ctttctcaacccaaggctcta